(19) United States
(12) Patent Application Publication
(10) Pub. No.: US 2024/0374700 A1
(43) Pub. Date: Nov. 14, 2024

(54) SYNTHETIC GLYCOCONJUGATE VACCINE PROTOTYPE AGAINST STREPTOCOCCUS SUIS

(71) Applicants: UNIVERSITÉ DE MONTRÉAL, Montréal (CA); UNIVERSITY OF ALBERTA, Edmonton (CA)

(72) Inventors: Mariela SEGURA, Saint-Hyacinthe (CA); Marcelo GOTTSCHALK, Saint-Hyacinthe (CA); Todd LOWARY, Edmonton (CA); Pei-Jhen LI, Edmonton (CA); Manas JANA, Edmonton (CA); Marco LO FIEGO, Edmonton (CA); Ryan SWEENEY, Edmonton (CA)

(21) Appl. No.: 18/704,158

(22) PCT Filed: Oct. 28, 2022
(86) PCT No.: PCT/CA22/51600

§ 371 Date: Apr. 24, 2024

Related U.S. Application Data
(63) Continuation of application No. 63/263,356, filed on Nov. 1, 2021.

(51) Int. Cl. A61K 39/09; A61K 39/00; A61K 39/385; A61P 31/04; C07H 15/04

(52) U.S. Cl. CPC A61K 39/092; A61K 39/385; A61P 31/04; C07H 15/04; A61K 2039/552; A61K 2039/55566; A61K 2039/6037; A61K 2039/6081

(57) ABSTRACT

The invention provides for a vaccine against S. suis serotype 2. The vaccine comprises chemically synthesized fragments and thus may be made widely commercially available. The vaccine is used in the livestock production and may be adapted for use against other serotypes of S. suis such as serotypes 1, 1/2, 3, 9, and 14. Also, the vaccine may be adapted for use in humans.

### FIELD OF THE INVENTION

The present invention relates generally to the prevention of diseases associated to Streptococcus suis (S. suis). More specifically, the invention relates to a vaccine against S. suis serotype 2. The vaccine comprises chemically synthesized fragments and thus may be made widely commercially available. The vaccine is used in the livestock production and may be adapted for use against other serotypes of S. suis such as serotypes 1, 1/2, 3, 9, and 14 as well as for use in humans.

### BACKGROUND OF THE INVENTION

Streptococcus suis (S. suis) is a major porcine pathogen that occurs worldwide and causes major economic losses. S. suis bacteria express capsular polysaccharides (CPS) a major bacterial virulence factor that defines the serotypes. S. suis can be transmitted to human beings by close contact with sick pigs or contaminated pork products. S. suis causes meningitis, septicaemia, endocarditis, arthritis, and septic shock in both pigs and human beings. The mortality in pigs is high. Currently, no effective vaccine to prevent S. suis-associated diseases in pigs is marketed. The use of autogenous vaccines in the field has been, so far, disappointing.

Autogenous vaccines consist of killed bacteria (“bacterin”) from the predominant strain(s) recovered in an affected farm, produced by licensed laboratories and given back to the same farm only. Consequently, these vaccines are “farm-specific”. Very few experimental studies showed that “laboratory-made” bacterins may be effective when combined with highly potent adjuvants. However, these combinations have been shown to be highly reactive. The resulting side-effects make such combinations commercially undesirable.

According to the Canadian Swine Health Information Network, S. suis-related diseases are the most common infectious problem reported in Canadian swine farms. In addition, after consulting in late 2018 with swine practitioners, the industry as well as expert bacteriologists and diagnosticians, the Monitoring and Analysis Working Group from the Swine Health Information Center (SHIC) in USA reviewed and established final rankings for what is now the Swine Bacteria Disease Matrix. As stated on its website, S. suis leads the list as the most important bacterial swine pathogen (https://www.swinehealth.org/swine-bacterial-disease-matrix/).

The incidence of the disease may be as high as 20%, although it is usually kept lower than 5% in the field, due to the extensive and routine prophylactic and metaphylactic use of antimicrobials.

It is highly desirable to reduce the use of antimicrobials in livestock production. S. suis disease prevention must focus instead on management of predisposal factors such as vaccination.

Previous S. suis vaccine work has focused on the evaluation of bacterins, subunit (mostly protein-based) vaccines or attenuated live bacterial cultures. For example, WO 2017/062558A1 [1] evaluates a bacterial CPS conjugate vaccine produced by a traditional method. The method uses a depolymerized portion of the native CPS (purified from bacterial culture) and conjugated to tetanus toxoid as carrier using the technique of reductive amination. This method is complex. Also, this method presents high batch-to-batch variability, difficulties in product characterization, and requires pathogen handling (level II) to produce the CPS.

The inventors are also aware of the document Zhang et al. [2], which discloses oligosaccharides antigens resembling the CPS of S. suis serotypes 2, 3, 9, and 14.

There is a need for effective vaccines against S. suis infections. There is a need for such vaccines that are commercially available for use by swine producers and veterinarians. Also, there is a need for such vaccines that may be adapted for use in humans.

### SUMMARY OF THE INVENTION

The inventors have designed and prepared a synthetic glycoconjugate vaccine comprising a fragment of the capsular polysaccharide (CPS) of serotype 2. The fragment is selected among fragments according to the invention, which are of different sizes and represent different antigenic epitopes of the CPS of serotype 2. Production of these fragments (compounds) comprises chemical and chemoenzymatic approaches.

It is known that the CPS consists of a linear core (backbone), functionalized with two different side-chain motifs. In embodiments of the invention, the fragments comprise the linear core alone. In other embodiments the fragments comprise one of the two side-chain motifs alone. In further embodiments, the fragments comprise a combination of the core and one of the two side-chain motifs. In further embodiments, the fragments comprise a combination of the core and the two side-chain motifs. In further embodiments, fragments according to the invention that contain the linear core may comprise 1-3 or more repeating units thereof.

In embodiments of the invention, each fragment is conjugated to a carrier protein. For example, CRM197 and BSA conjugates are prepared. As will be understood by a skilled person, other suitable carrier proteins may also be used. Such proteins include for example proteins from S. suis.

In further embodiments of the invention, a vaccine formulation is prepared using an adjuvant. For example, TiterMax Gold® and Montanide ISA 61 VG are used as adjuvants. As will be understood by a skilled person, other suitable adjuvants may also be used.

In further embodiments, the vaccine may be adapted for use against other serotypes of S. suis including serotypes 1, 1/2, 3, 9, and 14. Also, the vaccine may be adapted for use in humans.

In further embodiments, a vaccine is made using an additional fragment as described in the document Zhang et al. [2].

The invention thus provides the following in accordance with aspects thereof:
(1) A compound of general formula selected from the group consisting of: A0, B0, C0, D0, E0, and F0 below

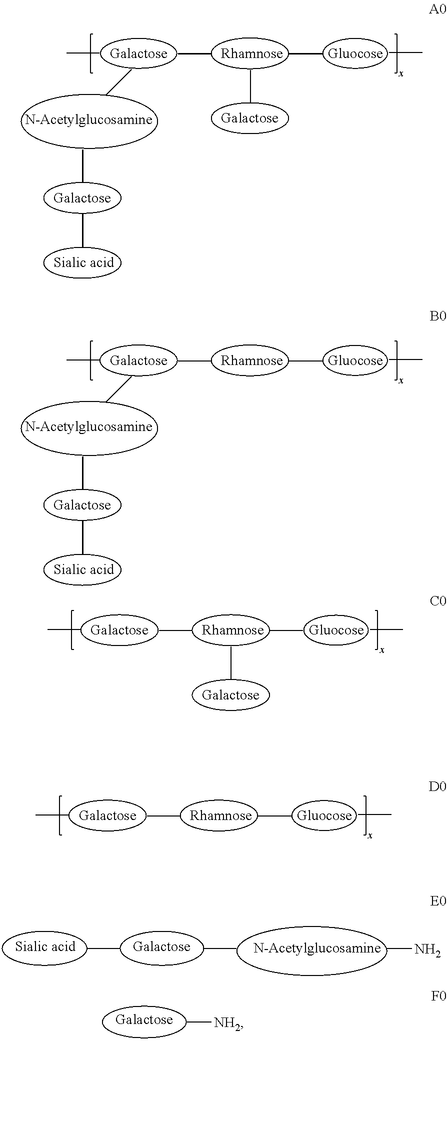

wherein:
x in A0-D0 is an integer selected from 1 to 6; preferably x is an integer selected from 1 to 3; and
a link between consecutive sugar moieties in the compound is a or p.
(2) A compound of general formula selected from the group consisting of: A, B, C, D, E, and F below

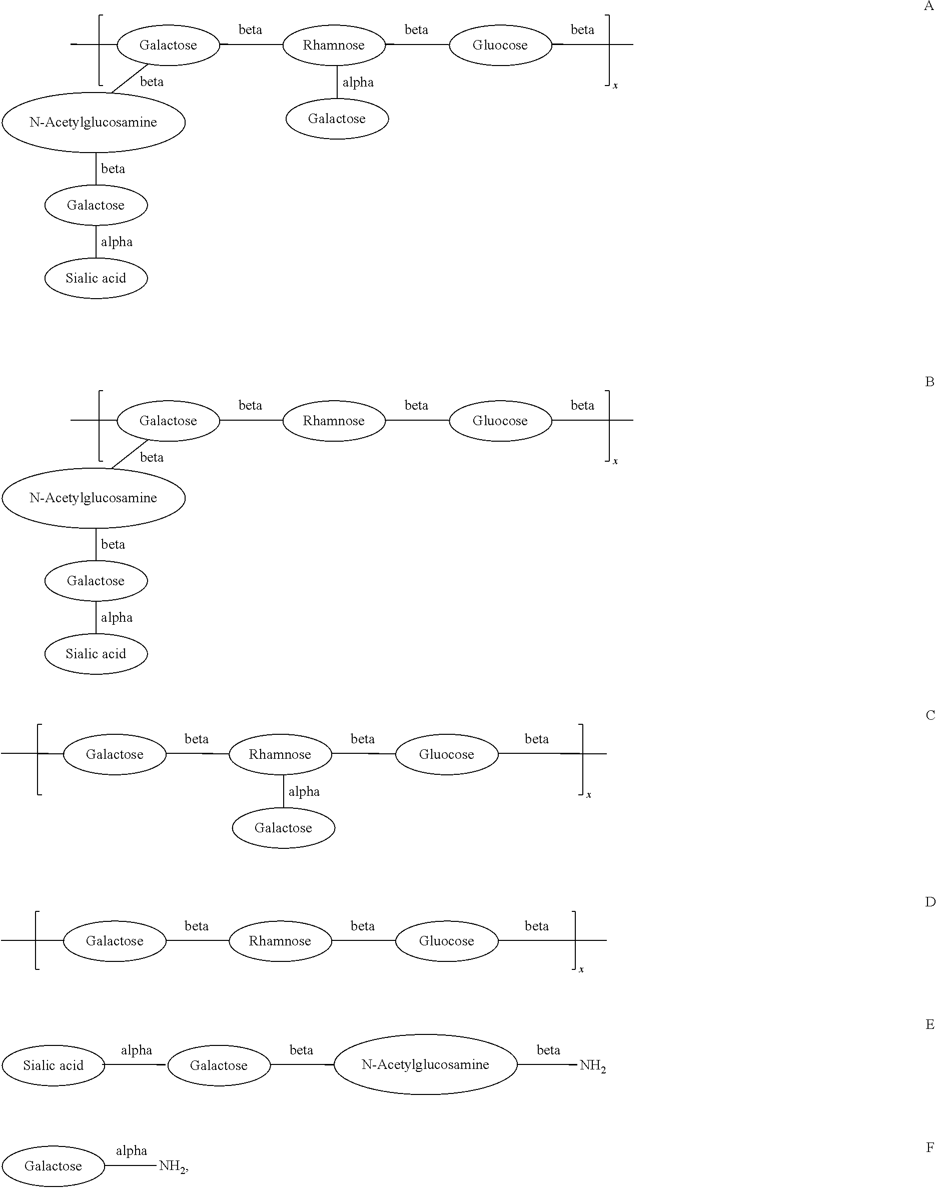

wherein x in A-D is an integer selected from 1 to 6; preferably x is an integer selected from 1 to 3.
(3) A compound of general formula selected from the group consisting of: A01, B01, C01, D01, E01, and F01 below

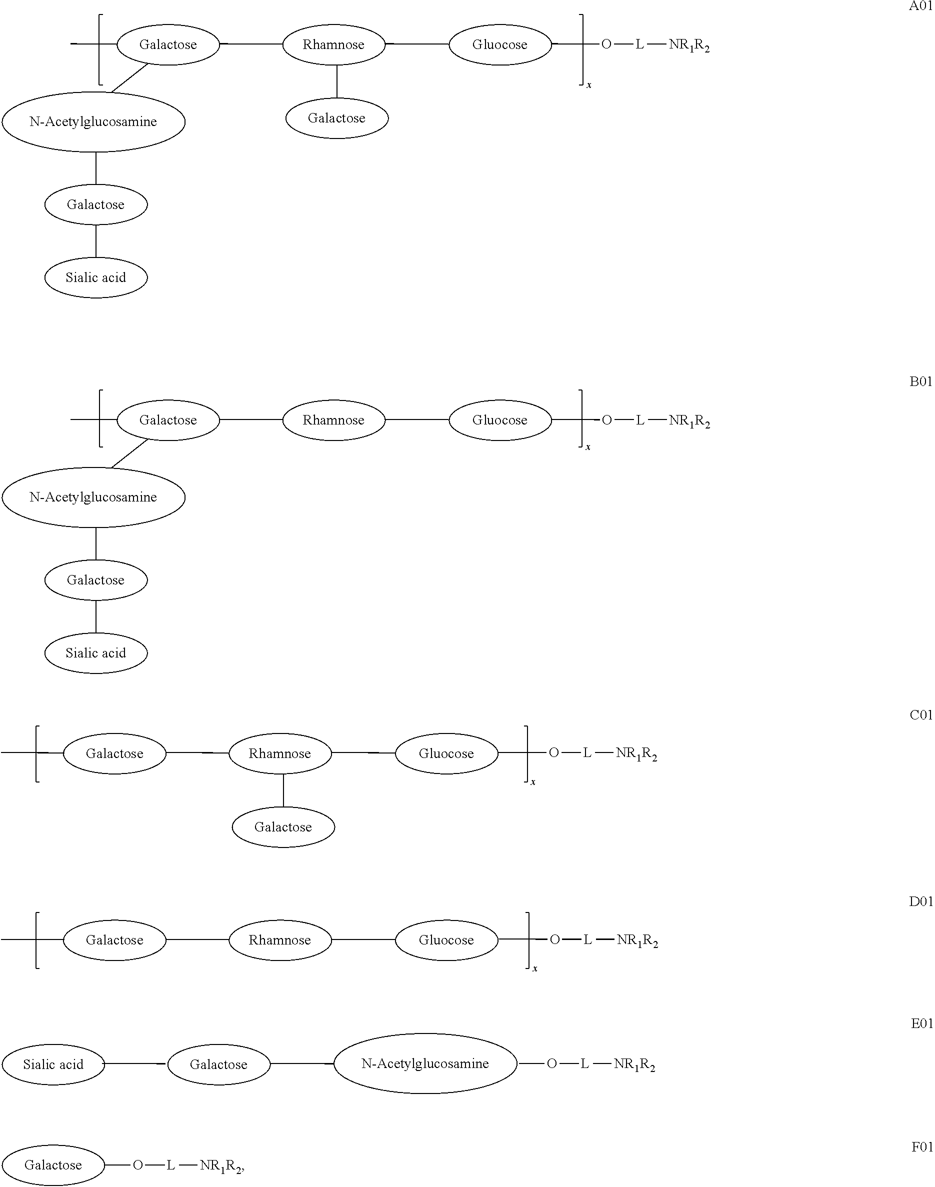

wherein:
x in A01-D01 is an integer selected from 1 to 6; preferably x is an integer selected from 1 to 3;
a link between consecutive sugar moieties in the compound is a or p;
L is a C1 to C12 linear, branched, or cyclic alkyl; preferably L is a linear alkyl; and
R₁ and R₂ are each independently H or a C1 to C6 linear, branched, or cyclic alkyl; or R₁ and R₂ together with N form a 3 to 6-member ring.
(4) A compound of general formula selected from the group consisting of: A1, B1, C1, D1, E1, and F1 below

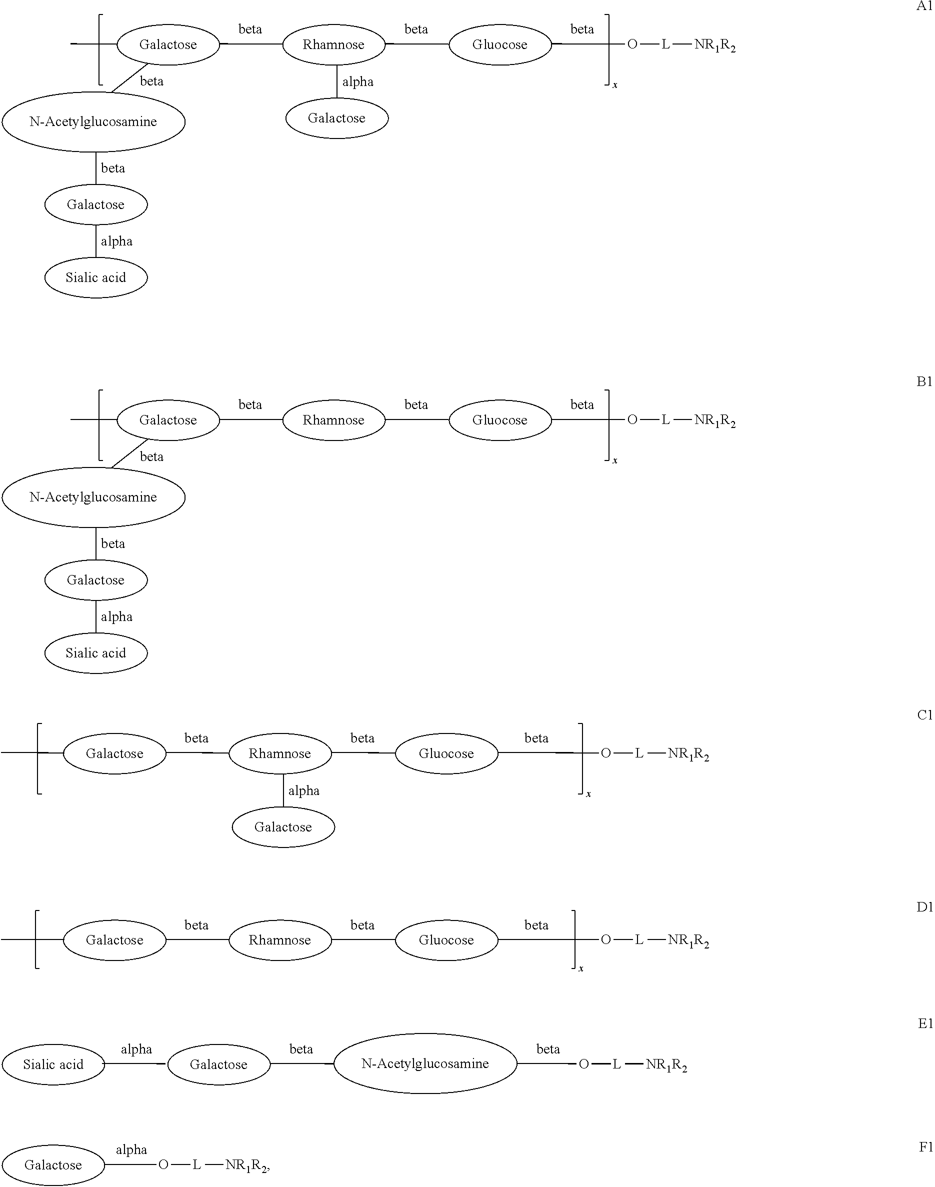

wherein:
x in A1-D1 is an integer selected from 1 to 6; preferably x is an integer selected from 1 to 3;
L is a C1 to C12 linear, branched, or cyclic alkyl; preferably L is a linear alkyl; and R₁ and R₂ are each independently H or a C1 to C6 linear, branched, or cyclic alkyl; or R₁ and R₂ together with N form a 3 to 6-member ring.
(5) A compound of general formula selected from the group consisting of: A02, B02, C02, D02, E02, and F02 below

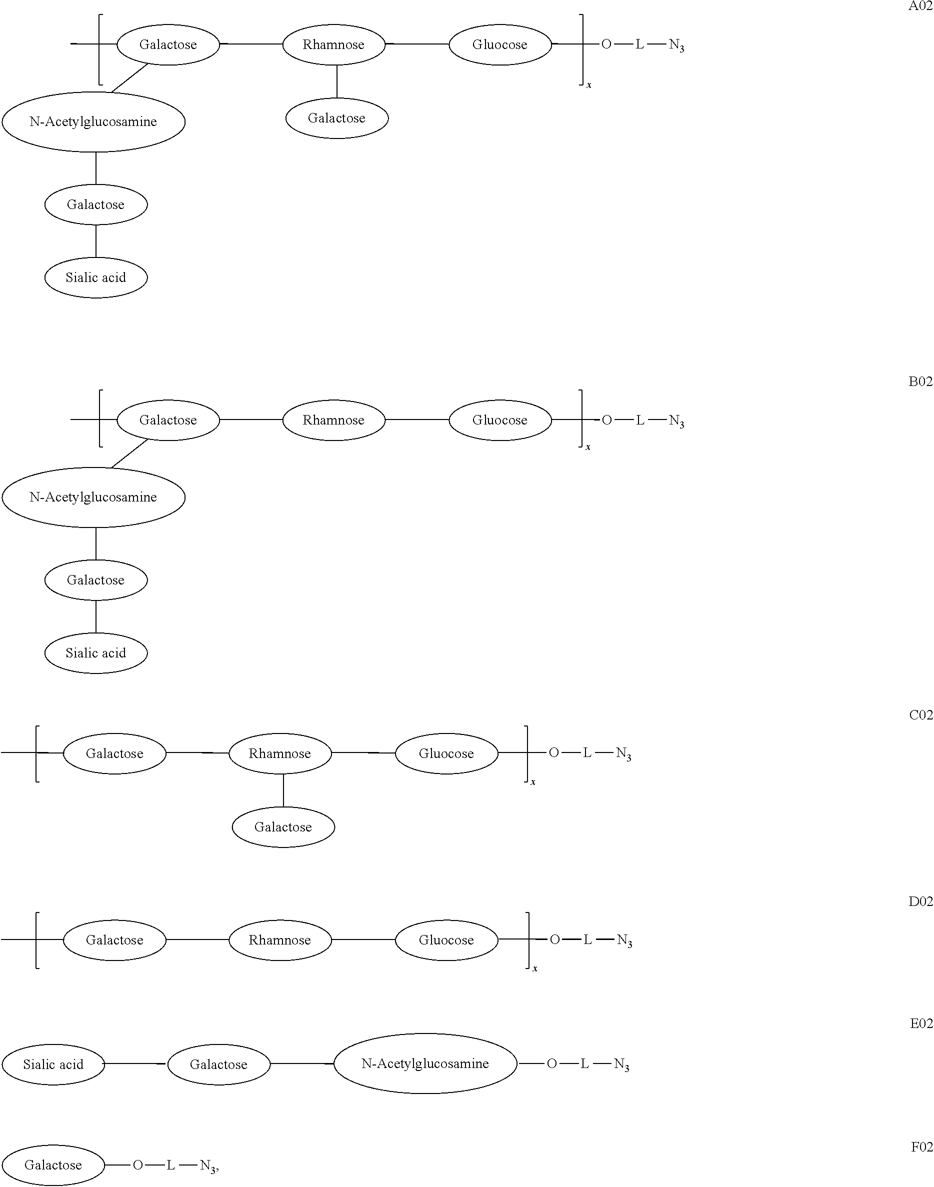

wherein:
x in A02-D02 is an integer selected from 1 to 6; preferably x is an integer selected from 1 to 3;
a link between consecutive sugar moieties in the compound is a or p;
L is a C1 to C12 linear, branched, or cyclic alkyl; preferably L is a linear alkyl; and R₁ and R₂ are each independently H or a C1 to C6 linear, branched, or cyclic alkyl; or R₁ and R₂ together with N form a 3 to 6-member ring.
(6) A compound of general formula selected from the group consisting of: A2, B2, C2, D2, E2, and F2 below

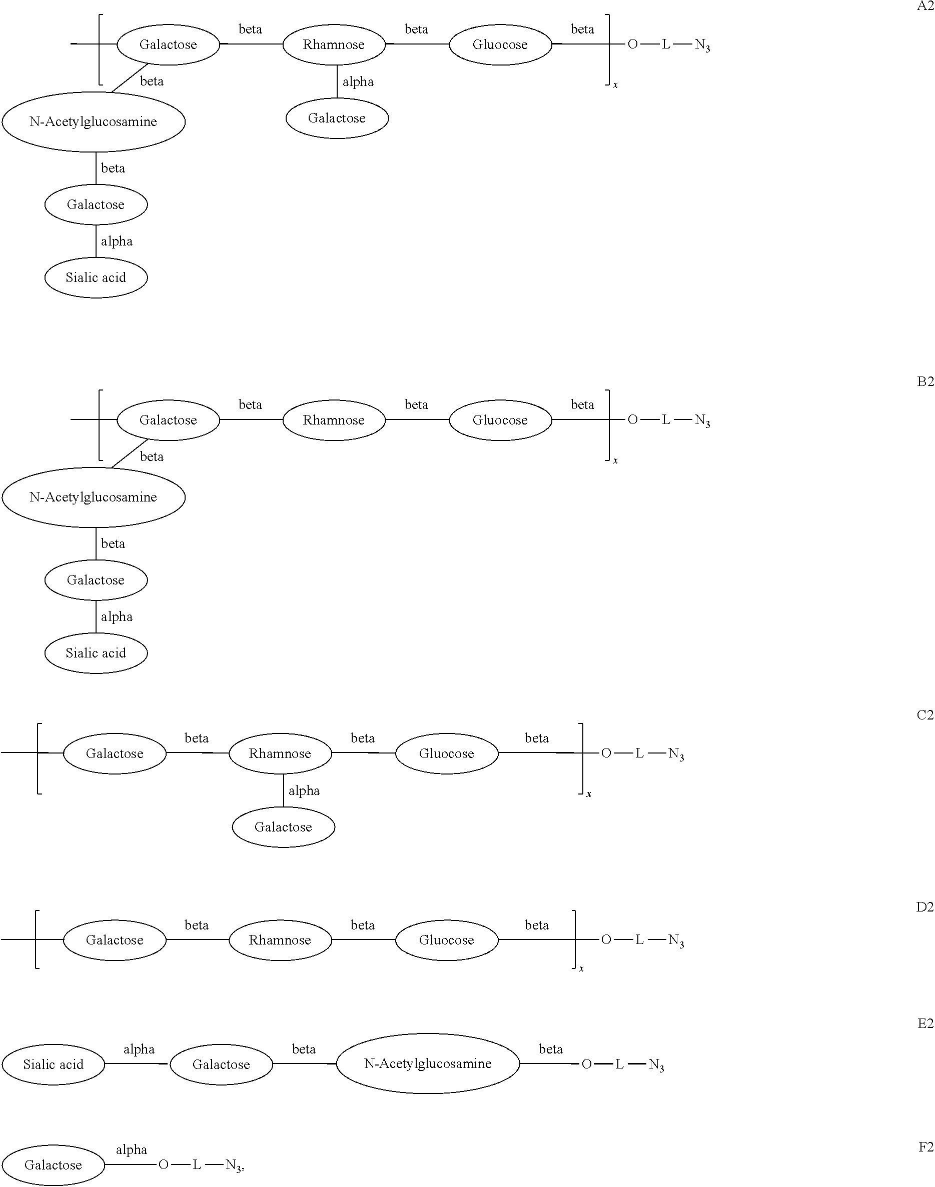

wherein:
x in A2-D2 is an integer selected from 1 to 6; preferably x is an integer selected from 1 to 3; and
L is a C1 to C12 linear, branched, or cyclic alkyl; preferably L is a linear alkyl.
(7) A compound selected from the group consisting of: 1, 4, 7, and 10-14 below

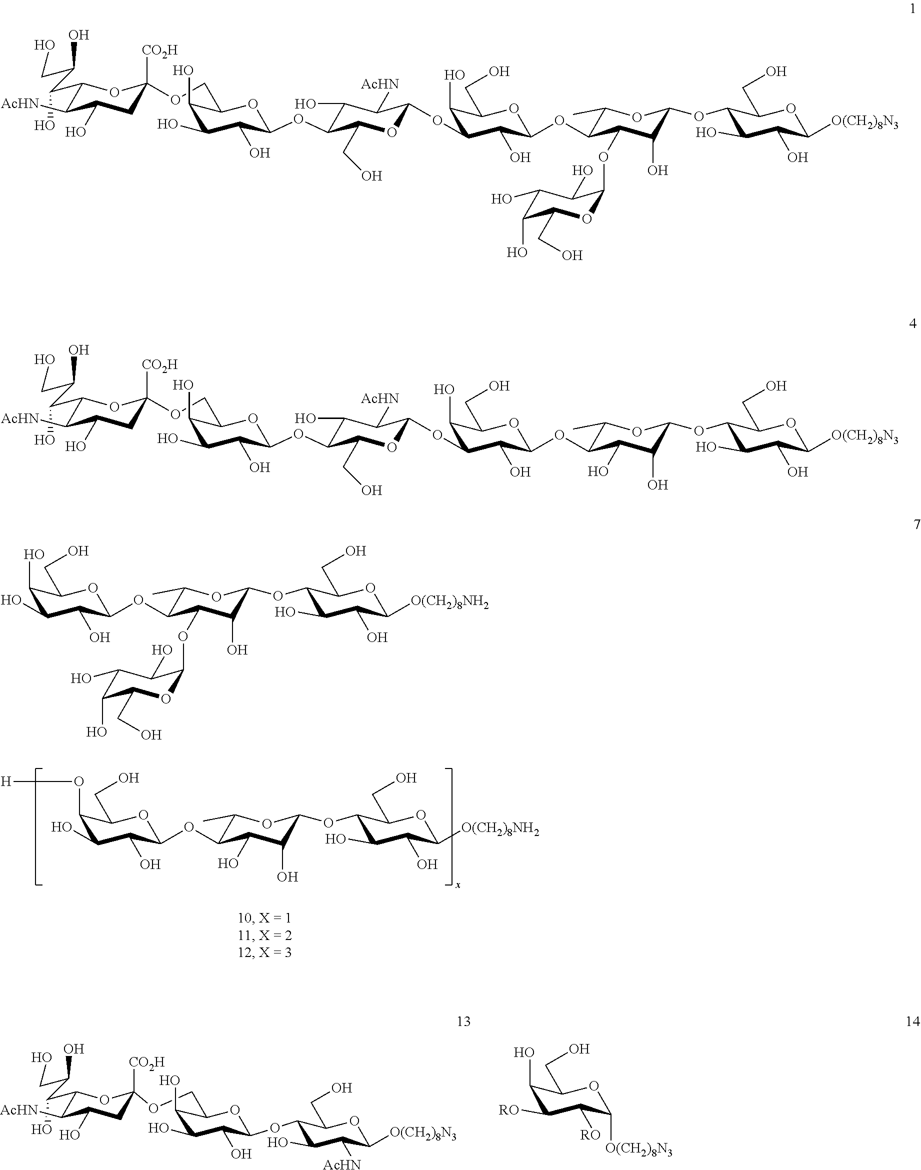

8. A compound selected from the group consisting of: 15, 16, 17, and 18-22 below

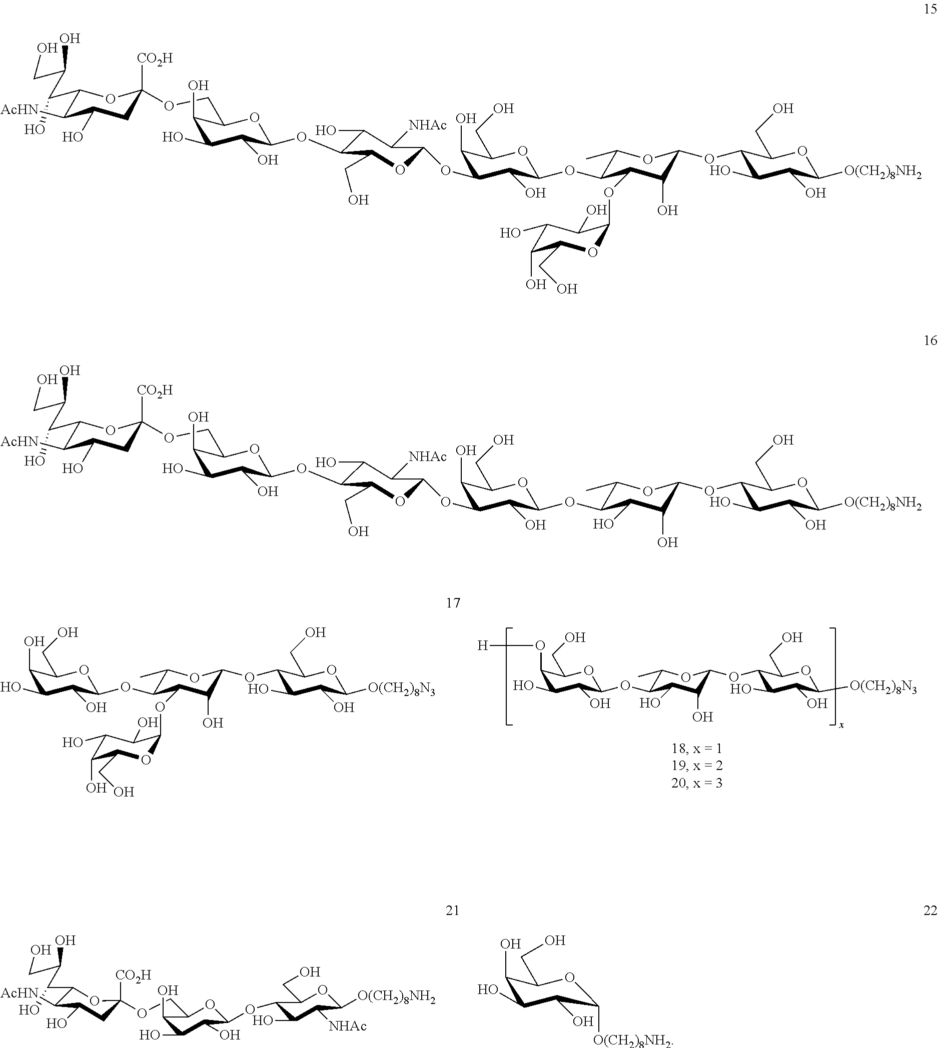

(9) A compound according to any one of (1) to (8) above, which is prepared by a process comprising: a chemical synthesis, a chemoenzymatic synthesis, or a combination of both chemical synthesis and chemoenzymatic synthesis.
(10) A glycoconjugate vaccine comprising a compound as defined in any one of (1) to (8) above, wherein the compound is conjugated with a carrier protein; preferably the carrier protein is CRM-197, BSA, a protein from Streptococcus suis (S. suis), or another suitable carrier protein.
(11) A vaccine formulation comprising a compound as defined in any one of (1) to (8) above and an adjuvant; preferably the adjuvant is TiterMax Gold®, Montanide™ ISA 61 VG, or another suitable adjuvant.
(12) A vaccine formulation comprising a glycoconjugate vaccine as defined in (10) above and an adjuvant; preferably the adjuvant is TiterMax Gold®, Montanide™ ISA 61 VG, or another suitable adjuvant.
(13) A vaccine formulation according to (11) or (12) above, which is commercially available.
(14) A vaccine formulation according to any one of (11) to (13) above, which is used in the production of livestock.
(15) A process for preparing a glycoconjugate vaccine as defined in (10) above or a vaccine formulation as defined in any one of (11) to (13) above, comprising: a chemical synthesis, a chemoenzymatic synthesis, or a combination of both chemical synthesis and chemoenzymatic synthesis.
(16) A method of preventing a disease associated to Streptococcus suis (S. suis) in a mammal, the method comprising administering to the mammal a compound as defined in any one of (1) to (8) above, a glycoconjugate vaccine as defined in (10) above, or a vaccine formulation as defined in any one of (11) to (13) above; preferably the disease is associated to a serotype of S. suis selected from the group consisting of serotypes 1, 1/2, 2, 3, 9, and 14; more preferably the disease is associated to serotype 2 of S. suis.
(17) Use of a compound as defined in any one of (1) to (8) above, a glycoconjugate vaccine as defined in (10) above, or a vaccine formulation as defined in any one of (11) to (13) above, for preventing a disease associated to Streptococcus suis (S. suis) in a mammal; preferably the disease is associated to a serotype of S. suis selected from the group consisting of serotypes 1, 1/2, 2, 3, 9, and 14; more preferably the disease is associated to serotype 2 of S. suis.
(18) Use of a compound as defined in any one of (1) to (8) above, in the manufacture of a vaccine for preventing a disease associated to Streptococcus suis (S. suis) in a mammal; preferably the disease is associated to a serotype of S. suis selected from the group consisting of serotypes 1, 1/2, 2, 3, 9, and 14; more preferably the disease is associated to serotype 2 of S. suis.
(19) A compound as defined in any one of (1) to (8) above, a glycoconjugate vaccine as defined in (10) above, or a vaccine formulation as defined in any one of (11) to (13) above, for use in the prevention of a disease associated to Streptococcus suis (S. suis) in a mammal; preferably the disease is associated to a serotype of S. suis selected from the group consisting of serotypes 1, 1/2, 2, 3, 9, and 14; more preferably the disease is associated to serotype 2 of S. suis.
(20) A method or use or compound for use according to any one of (16) to (19) above, wherein the mammal is human or non-human.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the appended drawings:

FIG. 1: Schematic representation of capsular polysaccharide (CPS) fragments (antigen) targets according to the invention. Monosaccharide definitions: Blue Circle=Glucose, Green Triangle=Rhamnose, Yellow Circles=galactose, Blue Square—N-Acetylglucosamine, Purple Diamond=Sialic Acid.

FIG. 2: Streptococcus suis serotype 2 CPS fragments 1, 4, 7, and 10-14.

FIG. 3: Antibody response against the corresponding synthesized CPS epitope of mice immunized with 25 μg of conjugate prototype 13 and prototype 14, formulated with TiterMax Gold (1/1 vol.). Mice (n=10 for each group, green dots) were immunized on day 0 and boosted on day 14. Placebo group of mice (n=5, black dots) received phosphate-buffered saline (PBS) formulated with the same adjuvant. Enzyme-linked immune assay (ELISA) plates were coated with corresponding synthetized prototype conjugated to bovine serum albumin (BSA) carrier protein and incubated with serum samples to measure anti-synthesized CPS epitope antibodies. Total (IgG plus IgM) antibody levels against the corresponding synthesized CPS epitope are shown for each mouse (one dot) with horizontal bars representing means (±SEM) of titer values. Statistical significance as follows: *, P<0.05; ***, P<0.001; ****, P<0.0001.

FIG. 4: Antibody response against the corresponding synthesized CPS epitope of mice immunized with 25 μg of conjugate prototype 7 and prototype 10, formulated with TiterMax Gold (1/1 vol.). Mice (n=10 for each group, green dots) were immunized on day 0 and boosted on day 14. Placebo group of mice (n=5, black dots) received PBS formulated with the same adjuvant. Naïve mice (n=5, white dots) were also included. ELISA plates were coated with corresponding synthetized prototype conjugated to BSA carrier protein and incubated with serum samples to measure anti-synthesized CPS epitope antibodies. Total (IgG plus IgM) antibody levels against the corresponding synthesized CPS epitope are shown for each mouse (one dot) with horizontal bars representing means (±SEM) of titer values. Statistical significance as follows: *, P<0.05; ***, P<0.001; ****, P<0.0001.

FIG. 5: Antibody response against the corresponding synthesized CPS epitope of mice immunized with 25 μg of conjugate prototype 1 and prototype 4, formulated with TiterMax Gold (1/1 vol.). Mice (n=10 for each group, green dots) were immunized on day 0 and boosted on day 14. Placebo group of mice (n=5, black dots) received PBS formulated with the same adjuvant. Naïve mice (n=5, white dots) were also included.

ELISA plates were coated with corresponding synthetized prototype conjugated to BSA carrier protein and incubated with serum samples to measure anti-synthesized CPS epitope antibodies. Total (IgG plus IgM) antibody levels against the corresponding synthesized CPS epitope are shown for each mouse (one dot) with horizontal bars representing means (±SEM) of titer values. Statistical significance as follows: **, P<0.01; ***, P<0.001. D: means “day”.

FIG. 6: Antibody response against native purified CPS (S. suis serotype 2) of mice immunized with 25 μg of conjugate prototype 13 and prototype 14, formulated with TiterMax Gold (1/1 vol.). Mice (n=10 for each group, green dots) were immunized on day 0 and boosted on day 14. Placebo group of mice (n=5, black dots) received PBS formulated with the same adjuvant. ELISA plates were coated with native (serotype 2) CPS and incubated with serum samples to measure anti-CPS antibodies. Total (IgG plus IgM) antibody levels against native purified CPS are shown for each mouse (one dot) with horizontal bars representing means (±SEM) of titer values. Statistical significance as follows: *, P<0.05; **, P<0.01. D: means “day”.

FIG. 7: Antibody response against native purified CPS (S. suis serotype 2) of mice immunized with 25 μg of conjugate prototype 7 and prototype 10, formulated with TiterMax Gold (1/1 vol.). Mice (n=10 for each group) were immunized on day 0 and boosted on day 14. Placebo group of mice (n=5) received PBS formulated with the same adjuvant. Naïve mice (n=5) were also included. ELISA plates were coated with native (serotype 2) CPS and incubated with serum samples to measure anti-CPS antibodies. Total (IgG plus IgM) antibody levels against native purified CPS are shown for each mouse (one dot) with horizontal bars representing means (±SEM) of titer values. Statistical significance as follows: *, P<0.05; **, P<0.01. D: means “day”.

FIG. 8: Antibody response against native purified CPS (S. suis serotype 2) of mice immunized with 25 μg of conjugate prototype 1 and prototype 4, formulated with TiterMax Gold (1/1 vol.). Mice (n=10 for each group) were immunized on day 0 and boosted on day 14. Placebo group of mice (n=5) received PBS formulated with the same adjuvant. Naïve mice (n=5) were also included. ELISA plates were coated with native (serotype 2) CPS and incubated with serum samples to measure anti-CPS antibodies. Total (IgG plus IgM) antibody levels against native purified CPS are shown for each mouse (one dot) with horizontal bars representing means (±SEM) of titer values. Statistical significance as follows: *, P<0.05; **, P<0.01; ***, P<0.001. D: means “day”.

FIG. 9: Antibody response against the corresponding synthesized CPS epitope of pigs immunized with conjugate prototype 1, 4, 7, 10, 11, or 12, formulated with Montanide ISA 61 VG. Immunization was performed with two doses at 14 day-interval. Three-week-old piglets were assigned either to placebo (n=10), or vaccine (n=10) groups. The first dose of vaccine and corresponding placebo adjuvant was administered intramuscularly at three weeks of age and the second dose, two weeks later. ELISA plates were coated with corresponding synthetized prototype conjugated to BSA carrier protein and incubated with serum samples (Day 25) to measure anti-synthesized CPS antibodies. Total Ig (IgG+IgM), IgM, IgG1, and IgG2 antibody levels are shown for each pig (one dot) with horizontal bars representing means (±SEM) of titer values. Statistical significance vs. placebo as follows: *, P<0.05; **, P<0.01; ***, P<0.001; ****P<0.0001.

FIG. 10: Clinical scores of piglets vaccinated with conjugate 1, 4, 7, 10, 11, or 12, formulated with Montanide ISA 61 VG. Immunization was performed with two doses at 14 day-interval. Three-week-old piglets were assigned either to placebo (n=10), or vaccine (n=10) groups. The first dose of vaccine and corresponding placebo adjuvant was administered intramuscularly at three weeks of age and the second dose, two weeks later. The challenge with 6 ml of 1.6×10⁹ CFU/ml of S. suis serotype 2, strain P1/7, was performed intraperitoneally at 11 days after the second vaccine dose. Clinical signs were monitored two to three times per day for up to ten days. Statistical significance vs. placebo: *, P<0.05. CNS: central nervous system.

FIG. 11: Survival rate of piglets vaccinated with conjugate 1, 4, 7, 10, 11, or 12, formulated with Montanide ISA 61 VG. Immunization was performed with two doses at 14 day-interval. Three-week-old piglets were assigned either to placebo (n=10), or vaccine (n=10) groups. The first dose of vaccine and corresponding placebo adjuvant was administered intramuscularly at three weeks of age and the second dose, two weeks later. The challenge with 6 ml of 1.6×10⁹ CFU/ml of S. suis serotype 2, strain P1/7, was performed intraperitoneally at 11 days after the second vaccine dose. Clinical signs were monitored two to three times per day for up to ten days. Statistical significance vs. placebo: *, P<0.05.

FIG. 12: Pig immunization with an “additional fragment” as proposed by Zhang et al. [2] and conjugated to CRM197. Immunization was performed with two doses (formulated with Montanide ISA 61 VG) at 14 day-interval. Three-week-old piglets were assigned either to placebo (n=15), or vaccine (n=15) groups. The first dose of vaccine and corresponding placebo adjuvant was administered intramuscularly at three weeks of age and the second dose, two weeks later. The challenge with 6 ml of 1.6×10⁹ CFU/ml of S. suis serotype 2, strain P1/7, was performed intraperitoneally at 11 days after the second vaccine dose. Clinical signs were monitored two to three times per day for up to ten days. A) Scheme of the “additional fragment”. B) Antibody response against the corresponding synthesized CPS epitope of pigs immunized with conjugated “additional fragment”. ELISA plates were coated with corresponding synthetized fragment conjugated to BSA carrier protein and incubated with serum samples to measure anti-synthesized CPS antibodies. Total Ig (IgG+IgM) antibody levels are shown for each pig (one dot) with horizontal bars representing means (±SEM) of titer values. Statistical significance vs. placebo as follows: *, P<0.05; ****P<0.0001. D: means “day”. C) Survival rate of piglets vaccinated with conjugated “additional fragment”, formulated with Montanide ISA 61 VG.

### DESCRIPTION OF ILLUSTRATIVE EMBODIMENTS

Before the present invention is further described, it is to be understood that the invention is not limited to the particular embodiments described below, as variations of these embodiments may be made and still fall within the scope of the appended claims. It is also to be understood that the terminology employed is for the purpose of describing particular embodiments; and is not intended to be limiting. Instead, the scope of the present invention will be established by the appended claims.

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this disclosure pertains.

Use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”), or “containing” (and any form of containing, such as “contain” and “contains”), are inclusive or open-ended and do not exclude additional, unrecited elements or process steps.

The terms “compound”, “fragment”, “conjugate prototype” and “prototype” are used interchangeably to refer to the compounds according to the invention.

The terms “pig” and “swine” are used interchangeably in the present specification.

The inventors have designed and prepared a synthetic glycoconjugate vaccine comprising a fragment of the capsular polysaccharide (CPS) of serotype 2. The fragment is selected among fragments according to the invention, which are of different sizes and represent different antigenic epitopes of the CPS of serotype 2. Production of the fragments (compounds) comprises chemical and chemoenzymatic approaches.

It is known that the CPS consists of a linear core (backbone), functionalized with two different side-chain motifs. In embodiments of the invention, the fragments comprise the linear core alone. In other embodiments the fragments comprise one of the two side-chain motifs alone. In further embodiments, the fragments comprise a combination of the core and one of the two side-chain motifs. In further embodiments, the fragments comprise a combination of the core and the two side-chain motifs. In further embodiments, fragments according to the invention that contain the linear core may comprise 1-3 or more repeating units thereof. In further embodiments, an additional fragment is synthetized based on the document Zhang et al. [2], in particular fragment number 4 thereof.

In embodiments of the invention, each fragment is conjugated to a carrier protein. For example, CRM197 and BSA conjugates are prepared. As will be understood by a skilled person, other suitable carrier proteins may also be used. Such proteins include for example proteins from S. suis.

In further embodiments of the invention, a vaccine formulation is prepared using an adjuvant. For example, TiterMax Gold® and Montanide ISA 61 VG are used as adjuvants. As will be understood by a skilled person, other suitable adjuvants may also be used.

In further embodiments, the vaccine may be adapted for use against other serotypes of S. suis including serotypes 1, 1/2, 3, 9, and 14. Also, the vaccine may be adapted for use in humans.

The following is a description of preferred embodiments of the invention.

### Example 1

Preparation of Structurally-Defined Fragments of the S. suis Capsular Polysaccharide (CPS) by Synthetic Chemistry

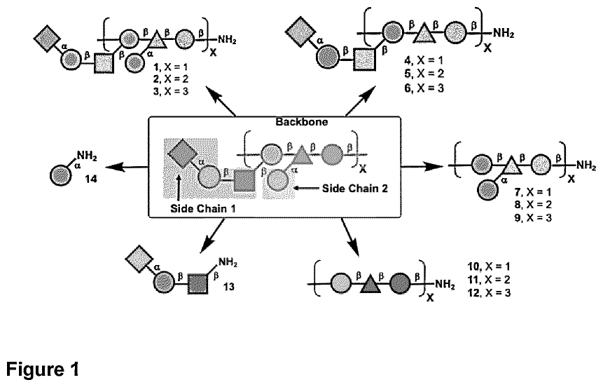

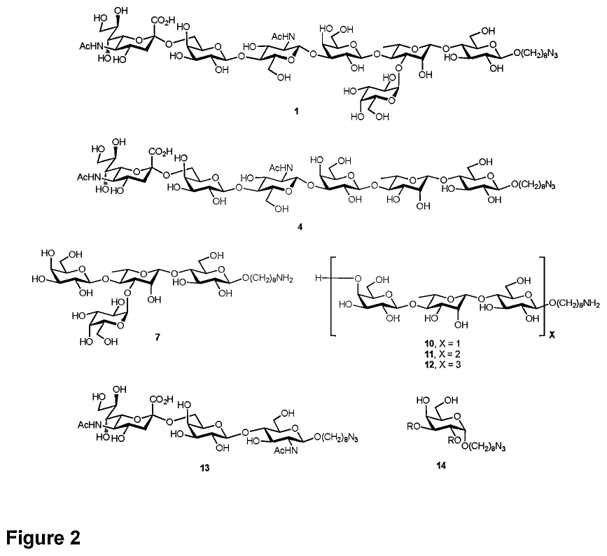

A schematic representation of the CPS fragments (antigen) targets is illustrated in FIG. 1. Eight fragments of the CPS from S. suis serotype 2, ranging in size from a monosaccharide to a heptasaccharide (1, 4, 7, and 10-14, FIG. 2) were selected for synthesis. These eight compounds were prepared bearing an 8-azidooctyl (or 8-aminooctyl) linker to facilitate their conjugation to proteins as required to elicit a robust immune response.

Synthesis of 1. The synthesis of 1 is provided in Schemes 1-3 below. It was first necessary to prepare three monosaccharides (1.4, 1.8, and 1.13, Scheme 1). The synthesis of 1.4 (Scheme 1A) started from the known monosaccharide 1.1 [3], which was converted into diol 1.2 in 73% yield upon treatment with benzaldehyde dimethyl acetal and camphorsulfonic acid. The two hydroxyl groups in 1.2 were then benzylated with benzyl bromide and sodium hydride thus providing 1.3 in 88% yield. Regioselective reductive ring opening of the benzylidene acetal ring in 1.3 upon reaction with triethylsilane and trifluoracetic acid afforded a 67% yield of alcohol 1.4.

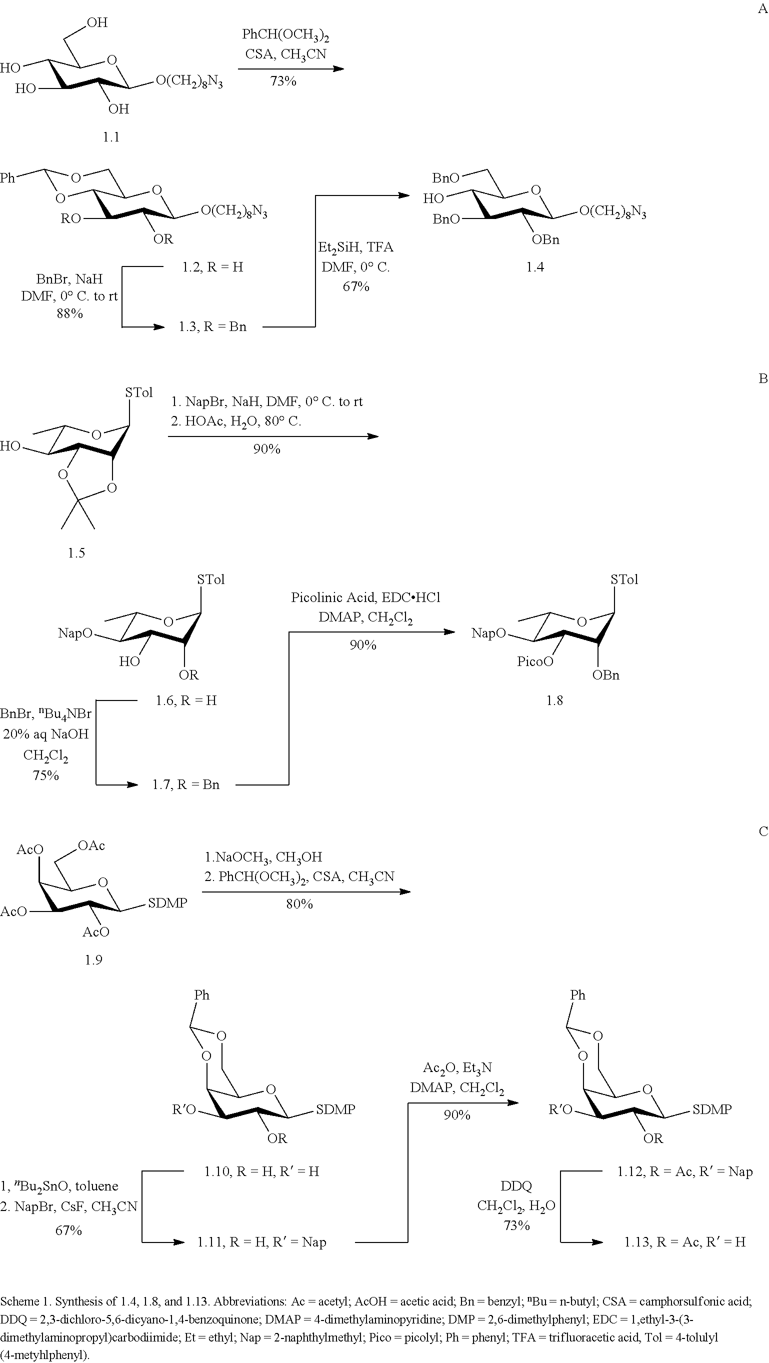

The synthesis of 1.8 (Scheme 1B) began with the known thioglycoside 1.5 [4], which, upon reaction with 2-naphtylmethyl bromide and sodium hydride and subsequent hydrolysis of the isopropylidene ketal, afforded a 90% of diol 1.6. The C-2 hydroxyl group was selectively benzylated under phase transfer conditions [5] providing a 75% overall yield of 1.7. Installation of a picolyl group was achieved, in 90% yield, by Steglich esterification using picolinic acid and 1-ethyl-3-(3-dimethylaminopropyl)-carbodiimide.

The previously reported 2,6-dimethylphenyl thioglycoside 1.9 [6] served as the starting material for the preparation of 1.13 (Scheme 1C). Removal of the acetyl groups using sodium methoxide and treatment of the resulting product with benzaldehyde dimethyl acetal and camphorsulfonic acid provided acetal 1.10 in 80% yield. The C-3 hydroxyl group in 1.10 was protected selectively as a naphthylmethyl ether by formation of an intermediate stannylidene acetal that was treated with 2-naphtylmethyl bromide. The product, 1.11, was formed in 67% yield over the two steps. Acetylation of the remaining hydroxyl group with acetic anhydride and pyridine proceeded in 90% yield to give 1.12. Cleavage of the naphthylmethyl ether upon treatment with 2,3-dichloro-5,6-dicyano-1,4-benzoquinone in a mixture of water and dichloromethane provided 1.13 in 73% yield.

With these monosaccharides in hand, they were used to assemble three disaccharide building blocks, 1.14, 1.17, and 1.18, as illustrated in Scheme 2. Disaccharide 1.14 was prepared as shown in Scheme 2A. Glycosylation of 1.4 with 1.8 was carried out under the promotion of N-iodosuccinimide and silver triflate starting the reaction at −15° C. and warming to 0° C. After the reaction separating the α/β isomers of the disaccharide was problematic; hence, the mixture was carried forward to the next step. Thus, the intermediate was treated with copper acetate in dichloromethane and methanol to cleave the picolyl ester. The expected product, 1.14, was obtained in 50% yield over the two steps. The ¹J_{C1,H1} of the rhamnose residue was 159.4 Hz, confirming the β-stereochemistry [7].

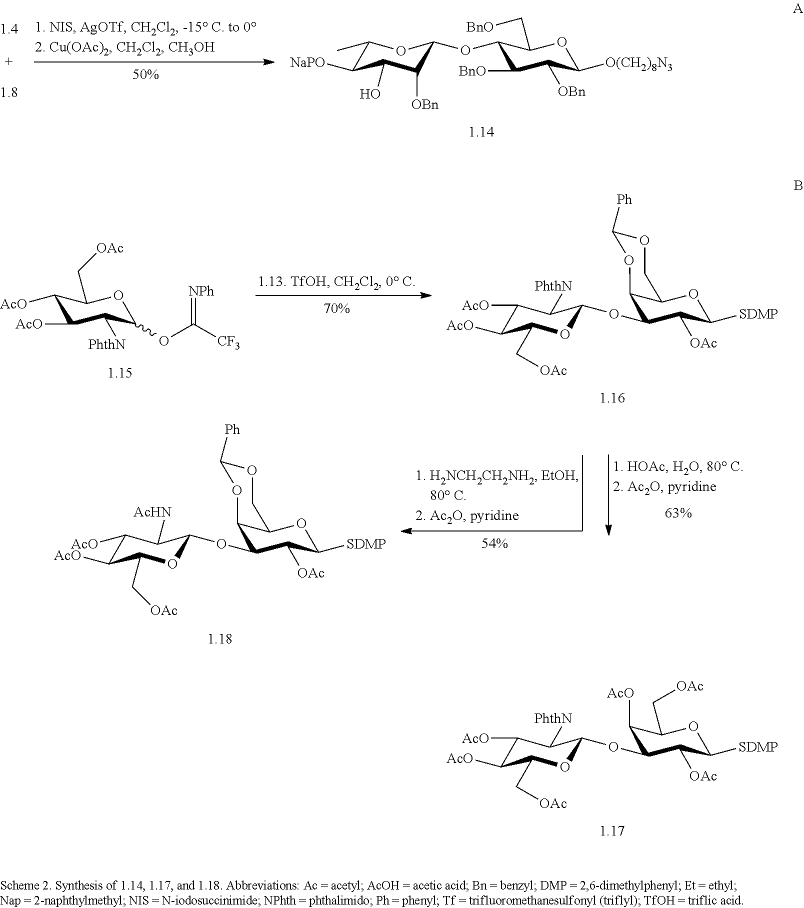

Scheme 2B illustrates the synthesis of disaccharide 1.17. Glycosylation of alcohol 1.13 with the known N-phenyltrifluoroacetimidate 1.15 [8] at 0° C. mediated by triflic acid provided the desired product 1.16 in 70% overall yield. The benzylidene acetal was cleaved by acid hydrolysis (heating in acetic acid/water) and the product diol was acetylated to give, over the two steps, a 63% yield of 1.17. In addition, 1.16 was converted, in 54% yield, to disaccharide 1.18, which was used in the synthesis of antigen 4 (see below). This was achieved by converting the phthalimido group to the N-acetate derivative in two steps: heating 1.16 in ethanol with ethylenediamine at reflux and acetylation of the product.

Having synthesized these disaccharides, they were converted to heptasaccharide 1 as shown in Scheme 3. Thioglycoside 1.19 [9] was used to glycosylate 1.14 upon treatment with N-iodosuccinimide and triflic acid. The product trisaccharide 1.19 was obtained in 84% yield. Oxidative cleavage of the naphthylmethyl ether in 1.20 was carried out by reaction with 2,3-dichloro-5,6-dicyano-1,4-benzoquinone in dichloromethane and water giving 1.21 in 73% yield. This alcohol was then glycosylated with thioglycoside 1.17 by treating them with N-iodosuccinimide and silver triflate, producing pentasaccharide 1.22 in 50% yield. The silyl acetal was removed by treatment with hydrofluoric acid in tetrahydrofuran and pyridine and then the phthalimido group was converted to the corresponding N-acetate derivative in two steps: heating 1.16 in ethanol with ethylenediamine at reflux and then acetylation of the product. The compound, 1.23, was obtained in 49% over the three steps from 12.1. At this point the protecting groups were removed in two steps. First, the benzyl ethers were oxidatively cleaved by treatment with potassium bromate in the presence of sodium dithionate in water and ethyl acetate [10]. Then, the acyl groups were cleaved by methanolysis via reaction with sodium methoxide in methanol. The deprotected pentasaccharide, 1.24, was obtained in 78% yield.

Compound 1.24 was then used in sequential glycosyltransferase-catalyzed reactions to install the final two carbohydrate residues. In the first of these reactions, a galactose residue was added using the HP0826 galactosyltransferase [11], which proceeded to give hexasaccharide 1.25 in 83% yield. The UDP-Gal donor required by the galactosyltransferase was prepared from galactose using the two enzymes: At USP and GalK [11]. The final monosaccharide residue adding using a mutant sialyltransferase (A200Y/S232Y Pd2,6ST) [12] and CMP-sialic acid. The mutant enzyme was required as use of the wild-type Pd2,6ST enzyme led to significant sialylation of the internal, as well as the terminal, galactose residue. The final heptasaccharide 1 was obtained in 95% yield.

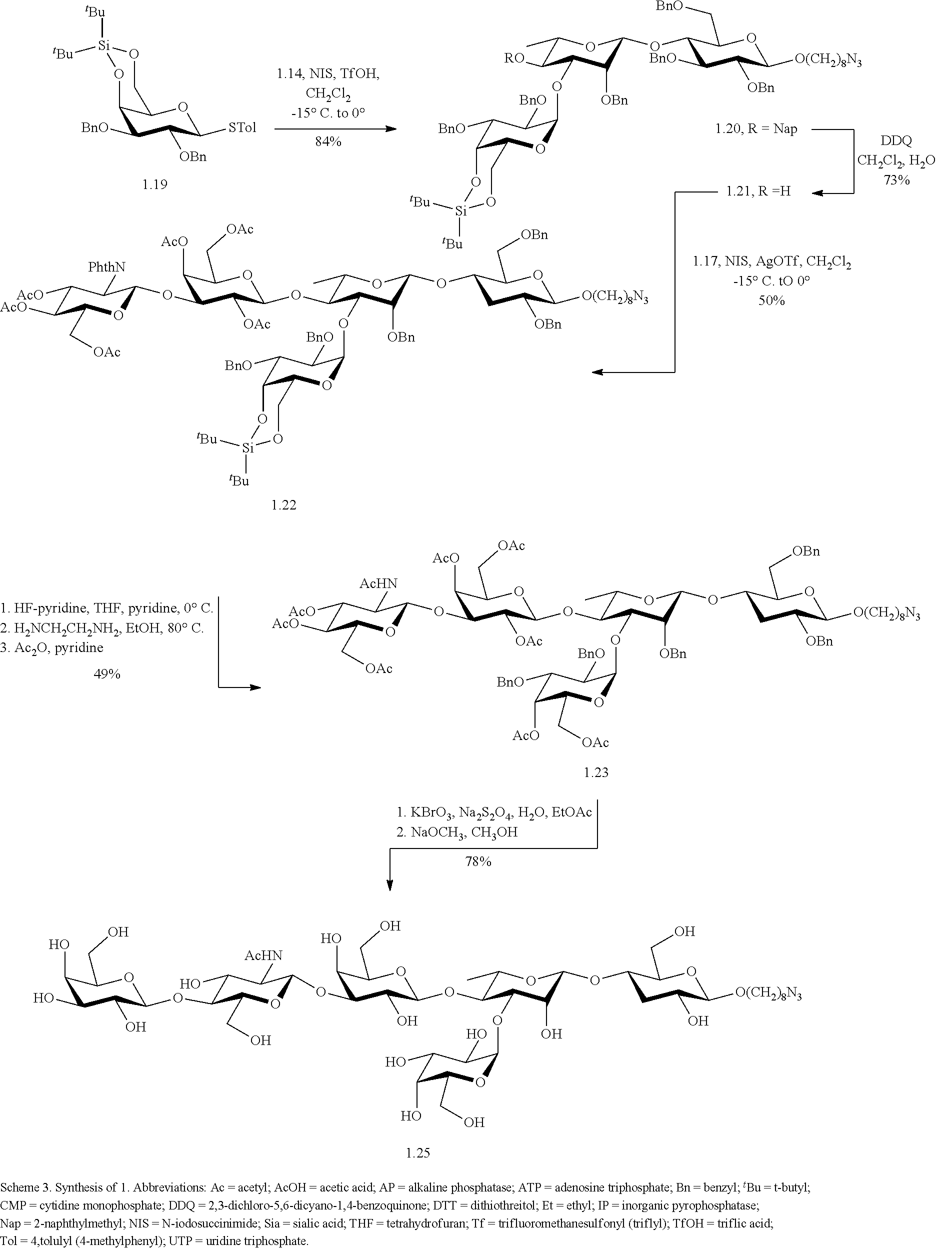

Synthesis of 4. The synthesis of 4 followed a similar path to 1 and is illustrated in Scheme 4 below. Starting with disaccharide 1.14, the alcohol was converted to the levulinate ester in quantitative yield upon treatment levulinic acid and 1-ethyl-3-(3-dimethylaminopropyl)-carbodiimide. The product, 4.1, was then converted to alcohol 4.2 by 2,3-dichloro-5,6-dicyano-1,4-benzoquinone-mediated cleavage of the naphthylmethyl ether affording a 77% yield of the desired compound. Glycosylation of 4.2 at 0° C. using disaccharide 1.18 promoted by N-iodosuccinimide and triflic acid provided a 51% yield of tetrasaccharide 4.3. Deprotection was achieved in three steps: acid hydrolysis of the benzylidene acetal, bromate-promoted oxidation of the benzyl ethers and finally deacylation. The product 4.5 was obtained in 71% yield over these three steps. From 4.5, the same enzymatic reactions used in the synthesis of 1 were employed. Thus, tetrasaccharide 4.5 could be converted to hexasaccharide 4 in two steps and 78% overall yield.

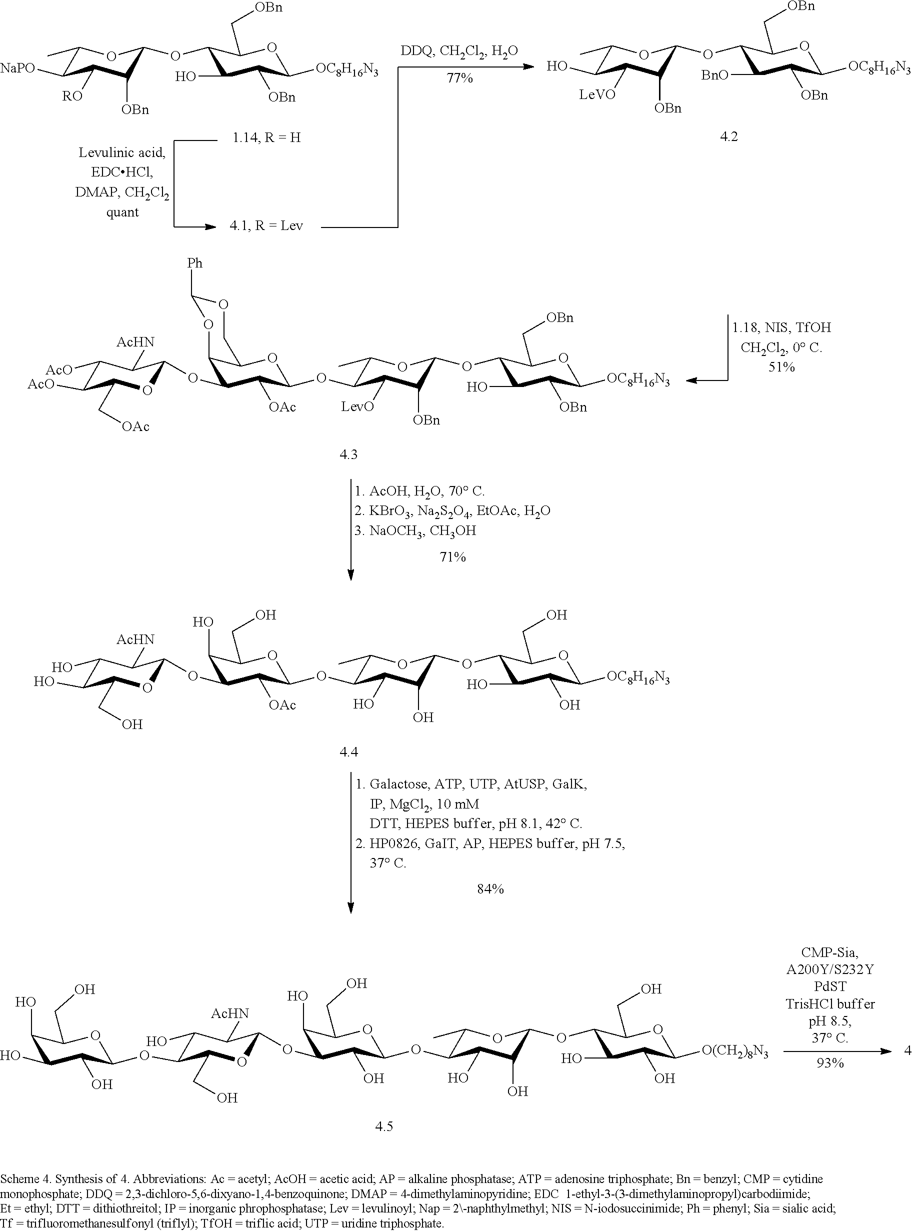

Synthesis of 7. To synthesize 7 (Scheme 5 below), thioglycoside 7.1 [13] was used to glycosylate trisaccharide alcohol 1.21 using N-iodosuccinimide and triflic acid starting at −15° C. and warming to 0° C. The expected tetrasaccharide 7.2 was obtained in 68% yield. Removal of the protecting groups was done in three steps. The silyl acetal was first cleaved by use of hydrofluoric acid-pyridine complex, and then the acetates were removed by methanolysis under basic conditions and finally the benzyl groups were removed by hydrogenolysis over palladium on carbon in methanol containing acetic acid. The latter step also reduced the azide to the amine. The product, 7, was obtained in 79% overall yield.

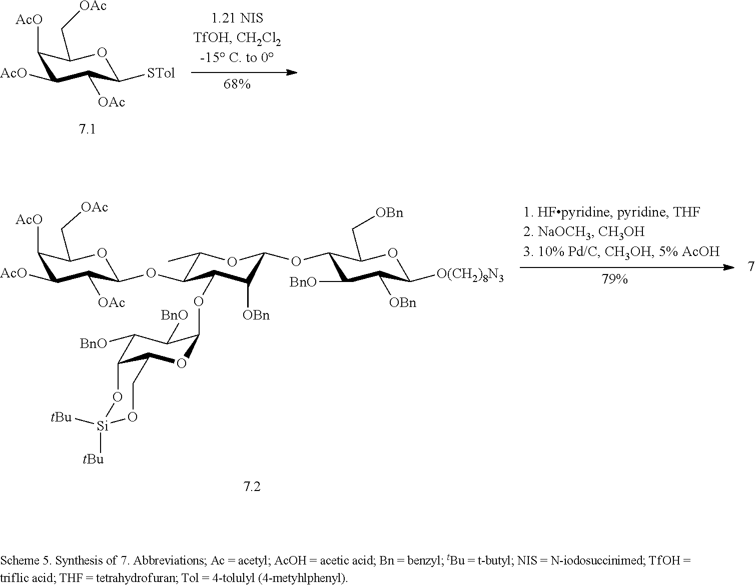

Synthesis of 10. Disaccharide 4.1 served as the starting point for the synthesis of 10 (Scheme 6 below). Oxidative cleavage of the naphthylmethyl ether as for the other compounds provided 10.1 in 70% yield. Glycosylation 10.1 with 7.1 [13] using N-iodosuccinimide and triflic acid afforded a 60% yield of trisaccharide 10.2. Deprotection of 10.2, and reduction of the azide to the amine, was carried out as for the synthesis of 7 to give trisaccharide 10 in 92% yield.

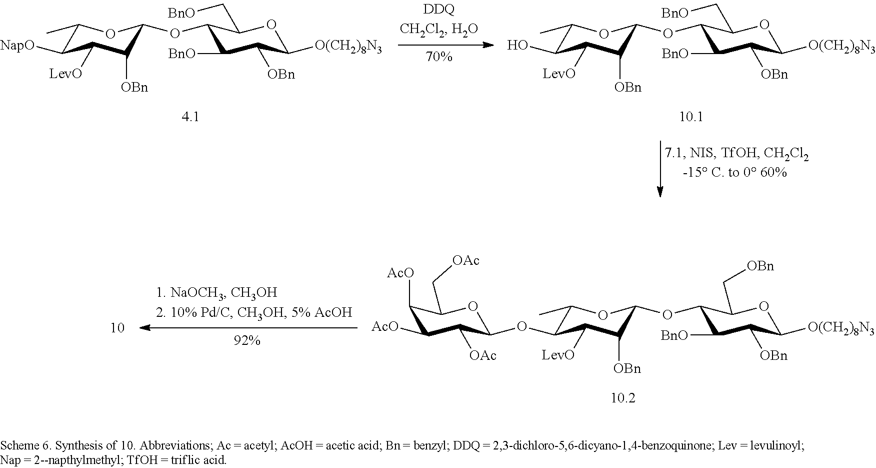

Synthesis of 11. Hexasaccharide 11 is a dimer of 10 and its synthesis required the preparation of two additional building blocks: monosaccharide 11.4 and disaccharide 11.9 (Scheme 7 below). The synthesis of 11.4 (Scheme 7A) started from the previously reported thioglycoside 11.1 [14]. The benzylidene acetal in 11.1 was hydrolyzed by heating with pyridinium p-toluenesulfonate and water in acetonitrile at reflux, providing diol 11.2 in 74% yield. The primary hydroxyl group was benzoylated with a limiting amount of benzoyl chloride in pyridine and then Steglich esterification of the product with levulinic acid and 1-ethyl-3-(3-dimethylaminopropyl)-carbodiimide afforded a 50% yield of 11.3. Hydrolysis of the thioglycoside with N-bromosuccinimide and water produced a hemiacetal that was converted to the corresponding N-phenyltrifluoroacetimidate upon reaction with N-phenyltrifluoroacetimidoyl chloride and cesium carbonate. The product, 11.4, was obtained in 90% yield over the two steps.

Synthesizing 11.9 (Scheme 7B) started with thioglycoside 1.8, which was hydrolyzed and converted in two steps and in 92% overall yield to N-phenyltrifluoroacetimidate 11.5, as done for the preparation of 11.4. This activatable donor was then used to glycosylate known alcohol 11.6. [15] The glycosylation was carried out using activation with triflic acid and the disaccharide product was immediately treated with copper acetate and water to remove the picolyl ester. This two-step transformation provided disaccharide alcohol 11.7 in 73% yield (¹J_{C1,H1} of rhamnose residue=158.8 Hz, confirming the β-stereochemistry). Benzoylation of the alcohol with benzoyl chloride and pyridine gave a near quantitative yield of 11.8. Conversion of this thioglycoside to N-phenyltrifluoroacetimidate 11.9 was done in 74% yield by treatment with N-iodosuccinimide and water and then reaction of the resulting product with N-phenyltrifluoroacetimidoyl chloride and cesium carbonate.

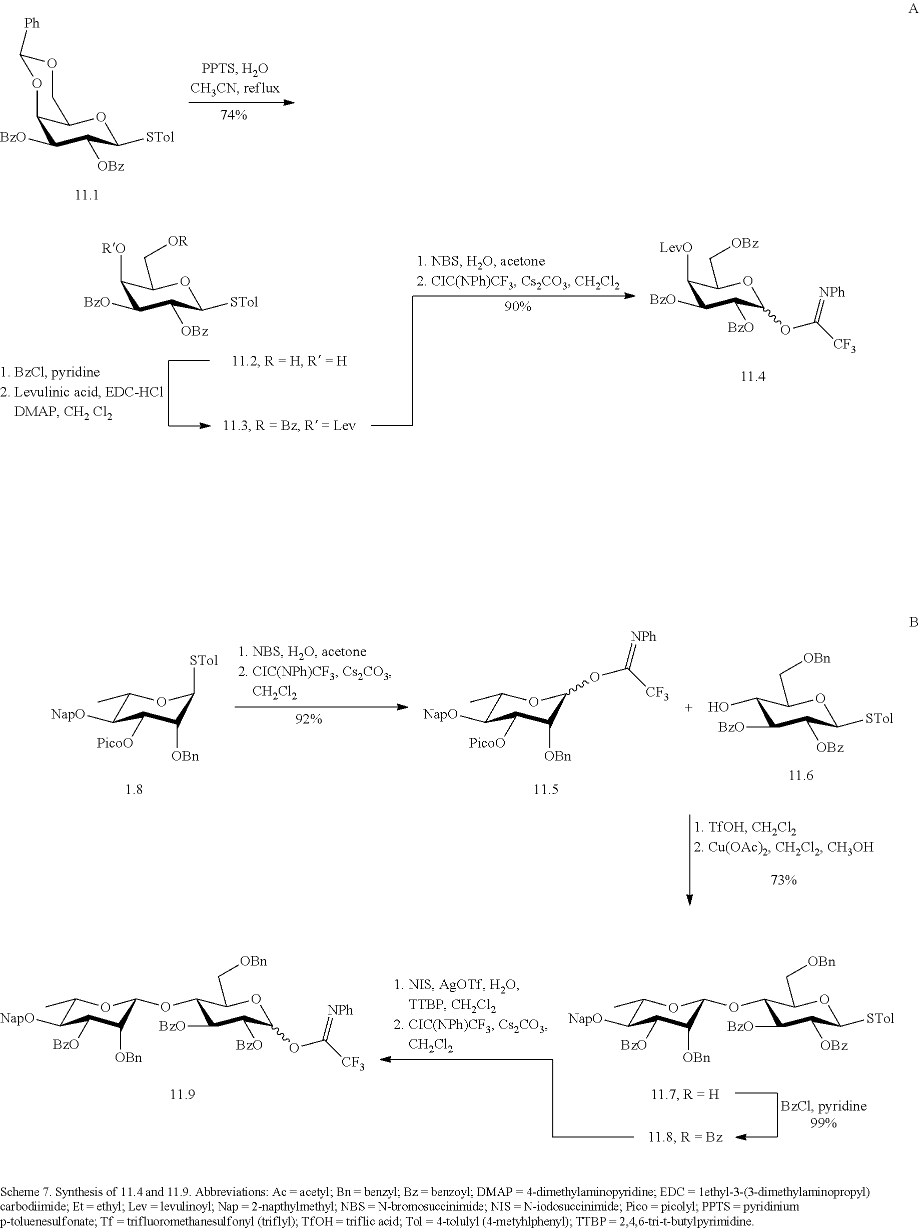

The synthesis of 11 is shown in Scheme 8 below. Disaccharide 1.14 was benzoylated with benzoyl chloride and pyridine to give 11.10 in 99% yield. The naphthylmethyl ether was cleaved by treatment with 2,3-dichloro-5,6-dicyano-1,4-benzoquinone and water in dichloromethane leading to the formation, in 80% yield, of alcohol 11.11. Glycosylation of 11.11 with N-phenyltrifluoroacetimidate 11.4 using triflic acid as the promotor provided an 80% yield of trisaccharide 11.12, which was then subjected to reaction with hydrazine hydrate to cleave the levulinate ester. The product alcohol 11.13 was obtained in 86% yield. Glycosylation of 11.13 with disaccharide N-phenyltrifluoroacetimidate 11.9 and then cleavage of the naphthylmethyl ether with 2,3-dichloro-5,6-dicyano-1,4-benzoquinone and water in the presence of β-pinene provided pentasaccharide 11.14 in 22% yield over the two steps. The poor step in this transformation was the glycosylation and all attempts to improve it failed. In addition, the product could not be purified after the glycosylation, but it could be after removal of the naphthylmethyl group. The β-pinene was added to the naphthylmethyl cleavage reaction as an acid scavenger to reduce the degree of undesired debenzylation [16]. Glycosylation of 11.14 with 11.4 promoted by triflic acid afforded hexasaccharide 11.15 in 60% yield. Finally, the compound was deprotected and the azide reduced as described for the synthesis of 7. The conversion of 11.15 into 11 proceeded in 92% overall yield over two steps.

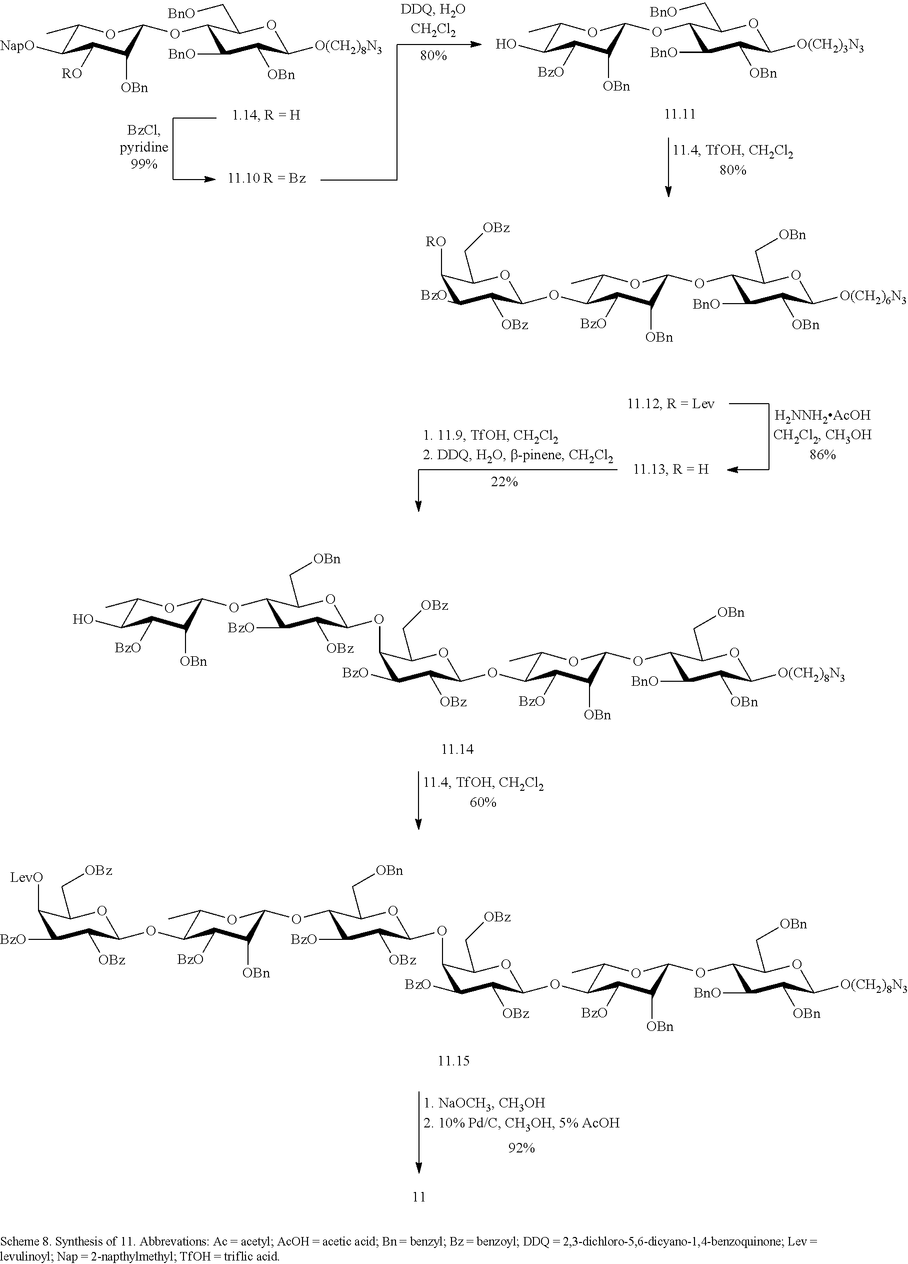

Synthesis of 12. The synthesis of 12 (Scheme 9 below) was achieved starting from 11.15, an intermediate used in the synthesis of 11. Cleavage of the levulinate ester in 11.15 with hydrazine acetate gave hexasaccharide alcohol 12.1 in 86% yield. Triflic acid-promoted glycosylation of 12.1 with disaccharide N-phenyltrifluoroacetimidate 11.9 was, like in the synthesis of 11.14, a poor reaction and purification could only be done after removal of the naphthylmethyl group. The product, 12.2 was obtained in 20% yield over the two steps. Glycosylation of 12.2 with 11.4 gave 12.3 (65% yield), which was then deprotected and converted to the amine in two steps as described for the synthesis of 7. Nonasaccharide 12 was obtained in 94% yield from 12.3.

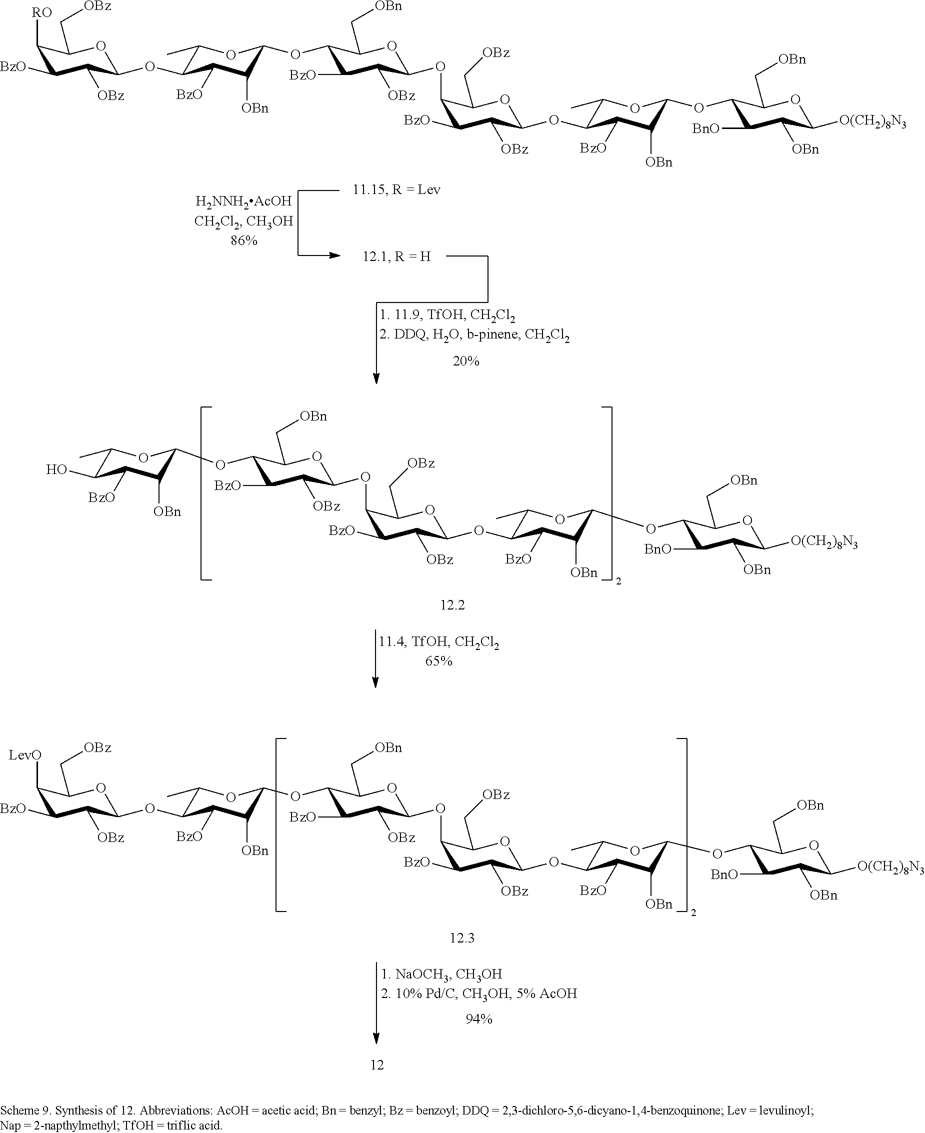

Synthesis of 13. The preparation of 13 was achieved in two enzymatic steps from the known 8-azidooctyl glycoside 13.1 [17] (Scheme 10 below) as was done for the other antigens. Galactosylation was mediated by the HP0826 galactosyltransferase and the sialylation used the native Pd2,6ST sialyltransferase. The product, 13, was obtained in 87% yield over the two steps.

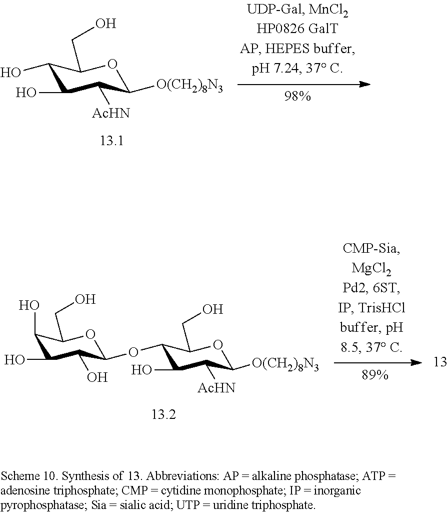

Synthesis of 14. Preparing 14 (Scheme 11 below) started from thioglycoside 14.1 [18], which was used to glycosylate 8-azido-octanol promoted by N-iodosuccinimide and triflic acid. The product, 14.2, was obtained in 60% yield. Deprotection was achieved in two steps. First, the silyl acetal was cleaved upon treatment with hydrogen fluoride-pyridine complex in a mixture of tetrahydrofuran and pyridine; this reaction gave 14.3 in 70% yield. The benzoate esters in 14.3 were then cleaved in 78% yield by methanolysis giving 14.

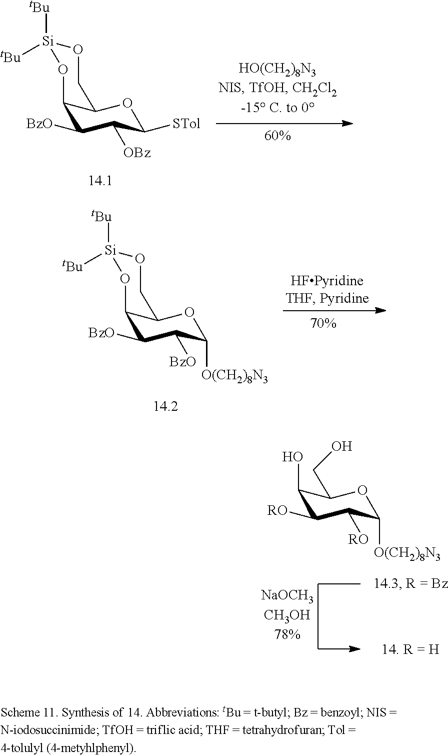

### Example 2

### Protein Conjugation (Using CRM197, a Non-Toxic Mutant of Diphtheria Toxin)

Preparation of CRM197 conjugates. To prepare conjugates of the antigens and CRM197 to be used in the immunizations, a maleimide-thiol coupling reaction was used (Scheme 12 below) [19]. Each antigen was converted to the corresponding amine by hydrogenation (Scheme 12A) and then coupled with 3,3′-dithiobis(sulfosuccinimidyl propionate) and then dithiothreitol to generate the corresponding 3′-thio-propylamide derivative (X—SH, e.g., 1-SH). Antigens 7 and 10-12 were generated as amines during final deprotection step and for these compounds the hydrogenation step was unnecessary. Separately, CRM197 was reacted with N-ε-maleimidocaproyl-oxysulfosuccinimide ester to produce the maleimide functionalized protein (Scheme 12B). The maleimide-containing CRM197 was then treated with an excess of the thiol-functionalized antigen (Scheme 12C). After the reaction, unreacted maleimide residues on the protein were capped with mercaptoethanol. Table 1 below shows the yields of the formation of X—SH and Table 2 below the loadings on the CRM-197 as determined by MALDI mass spectrometry.

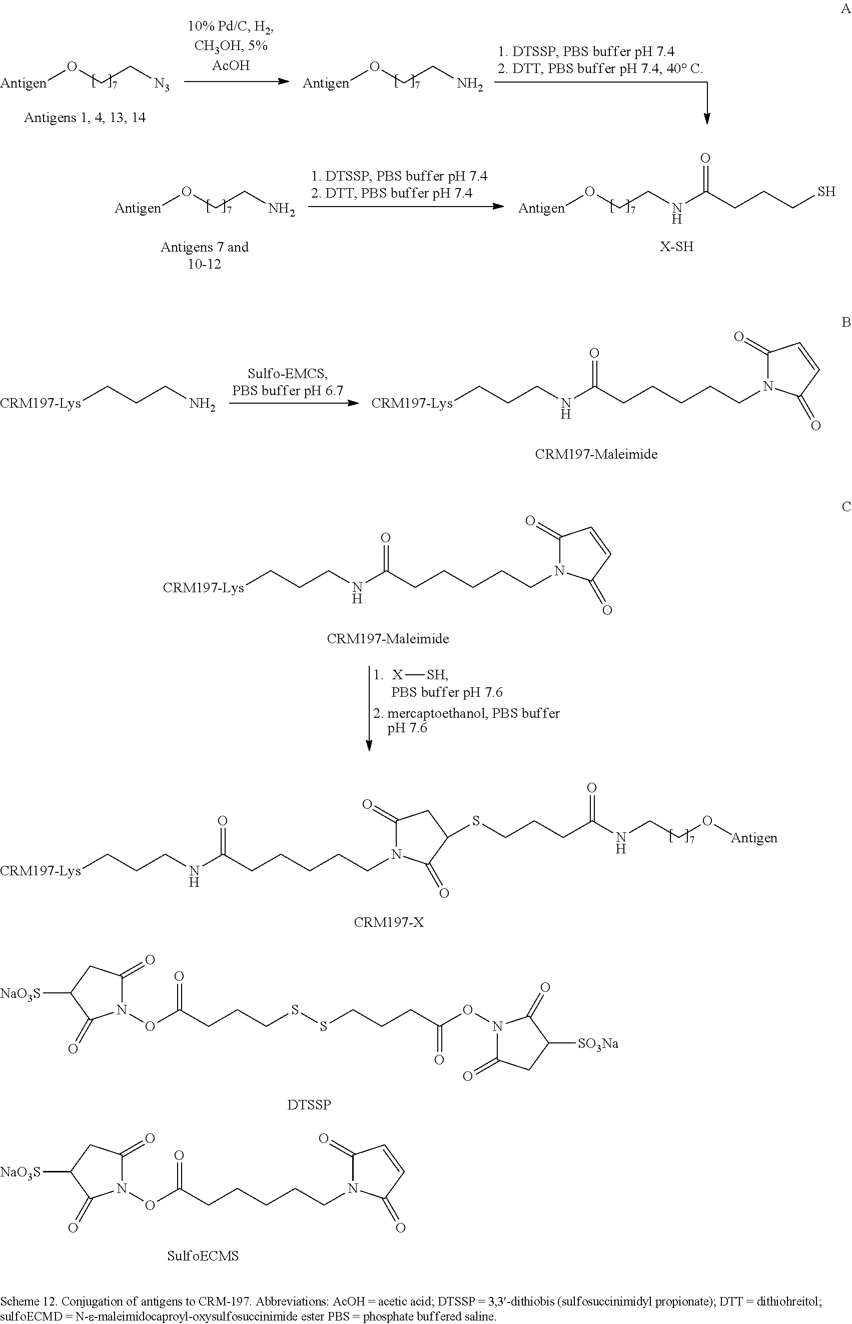

TABLE 1
Preparation of linker-functionalized antigens for CRM197
and BSA conjugate synthesis.

| | CRM197 Conjugates | | BSA Conjugates | |
|---|---|---|---|---|
| | Thiol | | Squaramide | |
| Compound | derivative | Yield | derivative | Yield |
| 1 | 1-SH | 77% | 1-SQ | 79% |
| 4 | 4-SH | 64% | 4-SQ | 75% |
| 7 | 7-SH | 60% | 7-SQ | 63% |
| 10 | 10-SH | 72% | 10-SQ | 71% |
| 11 | 11-SH | 88% | 11-SQ | 75% |
| 12 | 12-SH | 95% | 12-SQ | 77% |
| 13 | 13-SH | 90% | 13-SQ | 97% |
| 14 | 14-SH | 56% | 14-SQ | 82% |

TABLE 2
Preparation CRM197 and BSA conjugates and antigen loading
(from MALDI-MS).

| | CRM197 Conjugates | | | BSA Conjugates | |
|---|---|---|---|---|---|
| | Maleimide | Equivalents | Loading of | Equivalents | Loading |
| | Loading | thiol | Antigen | squaramide | of |
| Com- | on | derivative | on | derivative | Antigen |
| pound | CRM197 | used | CRM197 | used | on BSA |
| 1 | 27 | 75 | 5 | 75 | 6 |
| 4 | 25 | 75 | 6 | 75 | 6 |
| 7 | 24 | 100 | 15 | 100 | 9 |
| 10 | 19 | 100 | 9 | 100 | 13 |
| 11 | 29 | 35 | 10 | 50 | 7 |
| 12 | 21 | 50 | 6 | 50 | 5 |
| 13 | 24 | 200 | 9 | 100 | 11 |
| 14 | 19 | 200 | 9 | 100 | 15 |

Preparation of BSA conjugates. BSA conjugates of the antigens were also prepared for ELISA experiments. To control for any linker-specific antibodies generated during the immunizations, a different linker was used to prepare the BSA conjugates. A squaramide [20] was chosen for this purpose (Scheme 13 below). Each antigen was converted to the corresponding amine by hydrogenation (Scheme 13A) and then coupled with squaric acid dibutyl ester to generate the mono-squaramide derivative (X—SQ, e.g., 1-SQ). As was the case for the CRM197 conjugates, antigens 7 and 10-12 were generated as amines during final deprotection step thus the hydrogenation step was unnecessary. Coupling of each squaramide derivative with BSA afforded the conjugates. Table 1 above shows the yields of the formation of X—SQ and Table 2 above the loadings on the BSA as determined by MALDI mass spectrometry.

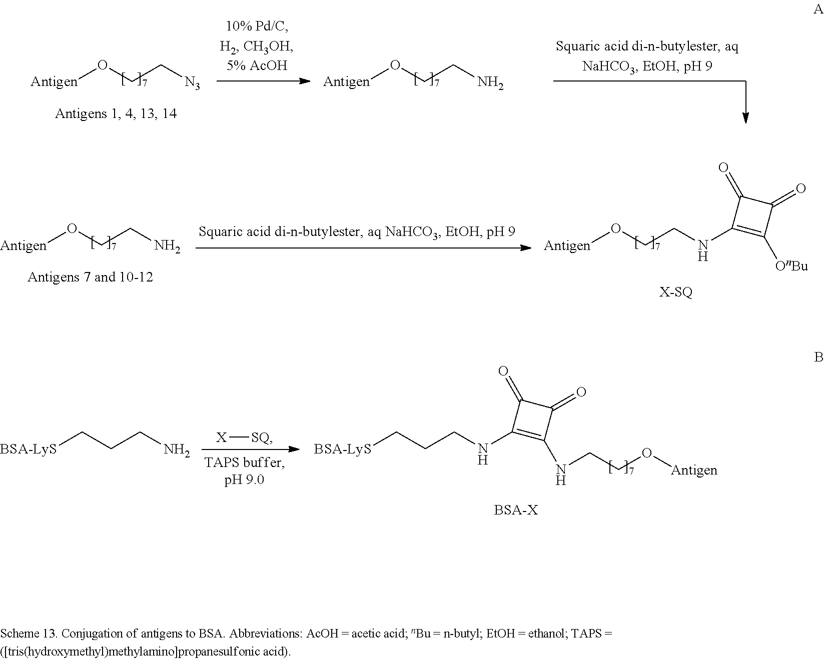

### Example 3

### Mouse Immunization

For mouse immunizations, prototypes 1, 4, 7, 10, 13, and 14 were used.

Vaccine formulation. The selected adjuvant for mouse immunization was TiterMax Gold® (an optimized adjuvant for mice; CytRx cooperation, Norcross, GA). The emulsion was prepared by mixing together the aqueous phase (PBS containing the vaccine antigen or prototype) with the oil phase (TiterMax Gold) in a 1:1 v/v ratio. For a vaccine dose of 25 μg given in 0.1 ml, the final concentration of the antigen in the final emulsion was 250 μg/ml. Therefore, the initial antigen concentration was equal to or greater than 0.5 mg/ml for the emulsion to work. When it was more concentrated, PBS was added to dilute the aqueous phase accordingly. After the first dose immunization, the emulsion was stored at 4° C. until the second dose (boost).

Mouse immunization and serum collection. Female, 5-week-old C57BL/6 mice (Charles River, Wilmington, MA) were used. Mice were immunized with two doses of 25 μg/100 μl volume subcutaneously at 14-day interval. First blood collection was performed from submandibular vein before second immunization (Day 14) and the final blood collection was performed by cardiac puncture 14 days after the last immunization dose (Day 28). A blood sample was also collected at Day 0 from naïve mice (to control antibody basal levels). Mouse groups were as follow: Naïve (n=5); Placebo (adjuvant only; n=5); and Conjugate (n=10).

Antibody titration. ELISA plates (Polysorp, Nunc-Immuno; Thermo Scientific, Mississauga, ON, Canada) were either coated with 100 μl of native purified S. suis type 2 CPS (diluted to 2 μg/ml in 0.1 M NaCO₃; pH 9.6), or with 100 μl of corresponding synthesized CPS epitope conjugated to BSA (diluted to 2 μg/ml in PBS; pH 7.4). Coated plates were left overnight at 4° C. For titration of mouse antibodies, coated plates were washed with PBS containing 0.05% (v/v) Tween 20 (PBS-T) and blocked by treatment with 300 μl of PBS containing 1% (w/v) bovine serum albumin (HyClone, Logan, UT) for 1 h at room temperature (RT). After washing, 100 μl of mouse serum samples serially diluted in PBS-T were added to the wells and left for 1 h at RT. After washing, antibodies were detected using either HRP-conjugated goat anti-mouse total Ig [IgG+IgM](Jackson ImmunoResearch), goat anti-IgG (Fcγ fragment specific; Jackson Immunoresearch), goat anti-IgM, goat anti-IgG1, goat anti-IgG2b or goat anti-IgG2c diluted at 1:1000, or goat anti-IgG3 diluted at 1:500 (Jackson Immunoresearch) for 1 h at RT. Plates were developed with 3,3′,5,5′-tetramethylbenzidine (TMB; InvitroGen, Burlington, ON, Canada) substrate and the enzyme reaction was stopped by addition of 0.5 M H₂SO₄. Absorbance was read at 450 nm with an ELISA plate reader.

For mouse serum titration, the reciprocal of the last serum dilution that resulted in an OD₄₅₀ of ≤0.15 (cutoff) was considered the titer of that serum. To control inter-plate variations, an internal reference positive control was added to each plate. For titration of mouse total Ig [IgG+IgM] antibodies, this control was a pool of hyper-immunized sera. The reaction in TMB was be stopped when an OD₄₅₀=1.0 was obtained for the positive internal control. For isotypes detection, the plates were stopped at 30 min.

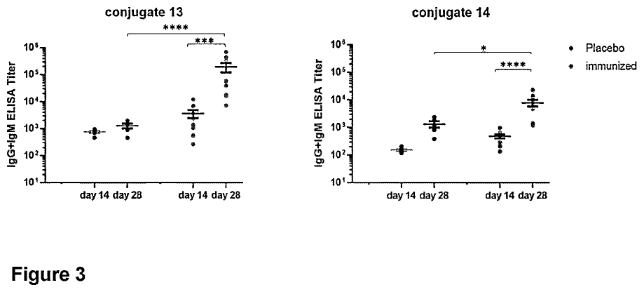

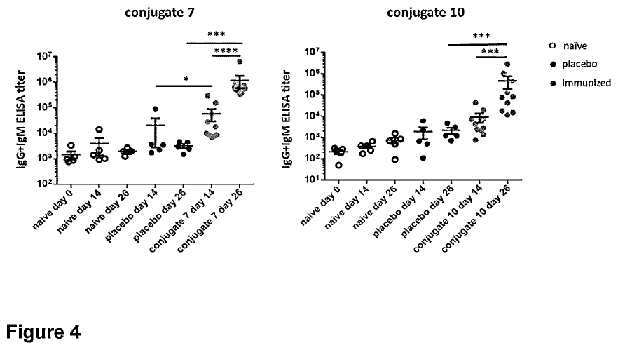

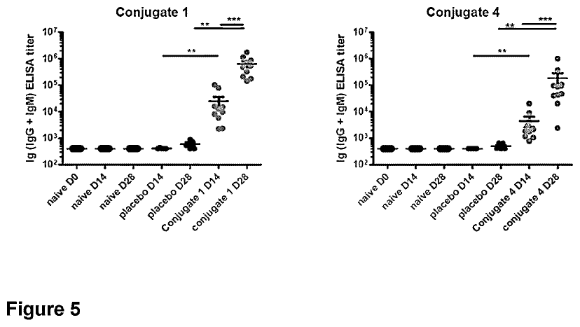

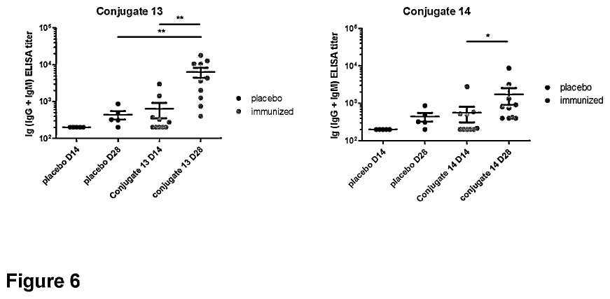

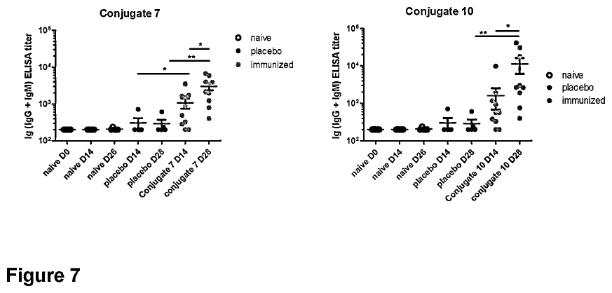

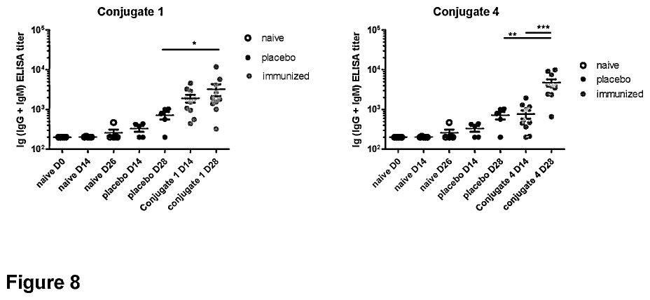

Experiments were conducted, showing total Ig (IgG+IgM) titers against the corresponding synthesized CPS epitope; see FIGS. 3-5. Further experiments were conducted, showing total Ig (IgG+IgM) titers against native purified CPS of S. suis serotype 2; see FIGS. 6-8. Based on antibody responses, conjugate prototypes 1, 4, 7, and 10 were selected for further studies in pigs.

### Example 4

### Pig Immunization

For pig immunizations, selected prototypes were: 1, 4, 7, 10, 11, and 12 were used. The “additional fragment” from the document Zhang et al. [2] was also used in pigs.

Vaccine formulation. The selected adjuvant for pig immunization was Montanide™ ISA 61 VG (SEPPIC, Fairfield, NJ), a water in oil (W/O) emulsion. Adjuvant to aqueous phase weight ratio is 60 g to 40 g for 100 g of a vaccine. Density of Montanide™ ISA 61 VG is 0.83. The vaccine dose was 170-200 μg/pig given in 1 ml. The vaccine formulation with Montanide™ ISA 61 VG was done according to the manufacturer's protocols.

Pig immunization and serum collection. Recently weaned, three-week-old, Landrace/white mixed breed piglets were acquired from a commercial farm in Quebec, with no history of clinical problems caused by S. suis, no vaccination program against this pathogen and free of Porcine Reproductive and Respiratory Syndrome virus. Upon arrival, piglets were weighed, individually tagged, assigned to two groups (placebo or vaccinated; n=10 or 15 per group) with equal average weight (approximately 5-6 kg), and placed in the Level II experimental animal facility of the Faculty of Veterinary Medicine, University of Montreal. Piglets were fed commercial, pelleted non-medicated food, with an addition of dry veggie supplements. Two days upon arrival, piglets were immunized intramuscularly (IM) in the neck muscle, with 1 ml of the selected vaccine prototypes (vaccine group) or adjuvant only in PBS (placebo control group). The second dose of vaccine and placebo were administered IM two weeks after the first dose. After the first-dose immunization, the vaccine emulsion was stored at 4° C. until the second dose. Blood samples were collected from the jugular vein before each immunization and before challenge for the determination of antibody responses (see below).

Pig challenge study and clinical scores. Eleven days after the second vaccine dose, the immunized and control animals were weighed, sedated using a dose of 0.5 mg/kg Atravet (Boehringer Ingelheim, Burlington, ON, Canada), and challenged with an intraperitoneal (IP) injection of 6 ml (9.6×10⁹ CFU) of a log-phase culture of S. suis serotype 2 strain P1/7. The average weight of the piglets on the day of the challenge was 14 kg. Following the challenge, pigs were monitored three times per day over a period of nine days for the presence of clinical signs and mortality. The individuals observing the animals were blinded to the treatments. A daily clinical score was calculated based on a clinical observation sheet. Assessed were general behavior, locomotion (musculoskeletal alterations) and functional alteration of the central nervous system (CNS). Behavior clinical scores were given as follows: 0=normal attitude and response to stimuli; 1=slight depression with marginally delay in the response to the stimuli, but preserved appetite; 2=moderate depression, animal only responds to repeated stimuli, reluctant to move, decreased appetite; 3=severe depression, non-responsive, recumbent, incoordination, diminished appetite. Locomotion clinical scores were given as follows: 0=normal gait and posture; 1=one joint affected, light lameness, but rises and moves without assistance; 2=moderate lameness, 2-3 joints affected with the swelling but stands without assistance; 3=severe lameness, ataxia 3-4 joints affected, recumbent and cannot stand or move.

Finally, CNS clinical scores were given as follows: 0=normal physiological behavior and response to stimuli; 1=slight incoordination, strabismus; 2=moderate incoordination, trembling; 3=sever, lateral or dorsal head inclination, ataxia, opisthotonus, nystagmus, convulsions. Pigs having a clinical score=3 in either category and a body temperature above 40° C. for two consecutive days were humanely euthanized. Ketamine (20 mg/kg; Narketan®, Vetoquinol, Lavaltrie, QC, Canada) and xylazine (2 mg/kg; Rompun®, Bayer, Mississauga ON, Canada) were administered IM to achieve complete anesthesia followed by intracardiac administration of pentobarbital sodium (100 mg/kg; Euthanyl®, Vetoquinol). Blood was collected from randomly selected piglets before euthanasia for bacteriological analyses (to confirm presence of the challenge strain). A post-mortem examination procedure was also conducted in selected animals. Swabs were collected from meninges and synovial fluid from affected joint cavities and seeded on blood agar for bacterial recovery. Samples of liver and spleen were collected and cultured for bacterial recovery. The individuals performing the necropsies and bacterial recovery were blinded to the treatments.

Antibody titration. ELISA plates (Polysorp, Nunc-Immuno; Thermo Scientific, Mississauga, ON, Canada) were either coated with 100 μl of native purified S. suis type 2 CPS (diluted to 2 μg/ml in 0.1 M NaCO₃; pH 9.6), or with 100 μl of corresponding synthesized CPS epitope conjugated to BSA (diluted to 2 μg/ml in PBS; pH 7.4). Coated plates were left overnight at 4° C. For titration of swine antibodies, coated plates were washed with PBS-T and blocked with 2% skim milk for 1 h at RT. To establish the antibody titers, pig sera were serially diluted (2-fold) in PBS-T (starting with a dilution of 1/200) and incubated for 1 h at RT. For titration of pig total Ig [IgM+IgG] or IgM, plates were incubated with peroxidase-conjugated goat anti-pig total Ig [IgM+IgG] diluted at 1:4000 (BioRad, Mississauga, Ontario) or anti-pig IgM diluted at 1:2000 (BioRad) for 1 h at RT. For porcine IgG1 or IgG2 detection, mouse anti-porcine IgG1 (diluted at 1:2000) or IgG2 (diluted at 1:2000) (BioRad) was added for 1 h at RT. After washing, peroxidase-conjugated goat anti-mouse Ig [IgM+IgG] diluted at 1:4000 (Jackson ImmunoResearch) was added for 1 h at RT. Plates were developed with TMB substrate, and the enzyme reaction was stopped by the addition of 0.5 M H₂SO₄. Absorbance was read at 450 nm with an ELISA plate reader. The reciprocal of the last serum dilution that resulted in an optical density at 450 nm (OD₄₅₀) of ≤0.2 (cutoff) was considered the titer of that serum. To control inter-plate variations, an internal reference positive control was added to each plate. This control was a convalescent serum from a S. suis experimentally infected pig. Reaction in TMB was stopped when an OD₄₅₀=1.0 was obtained for the positive internal control.

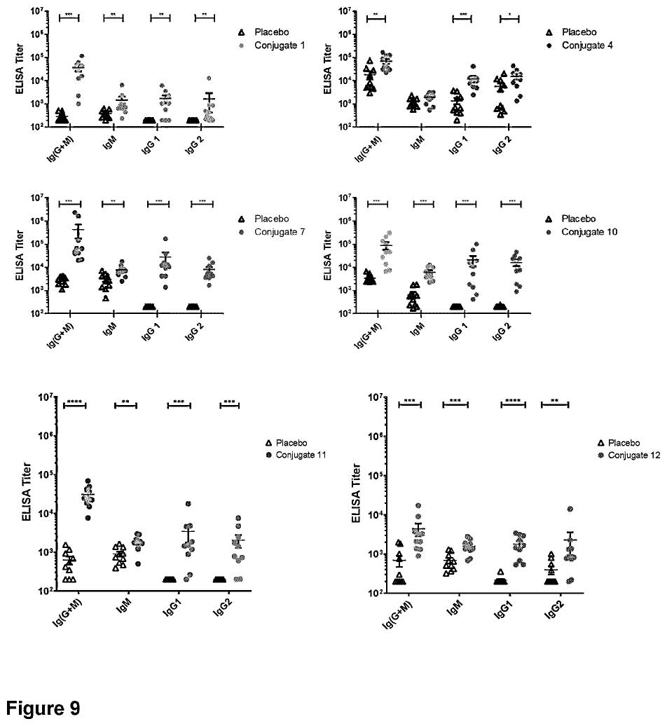

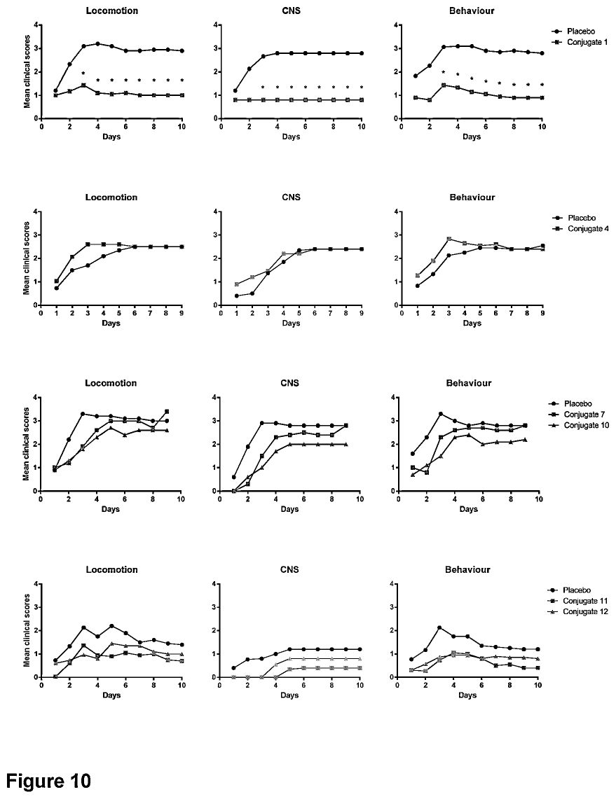

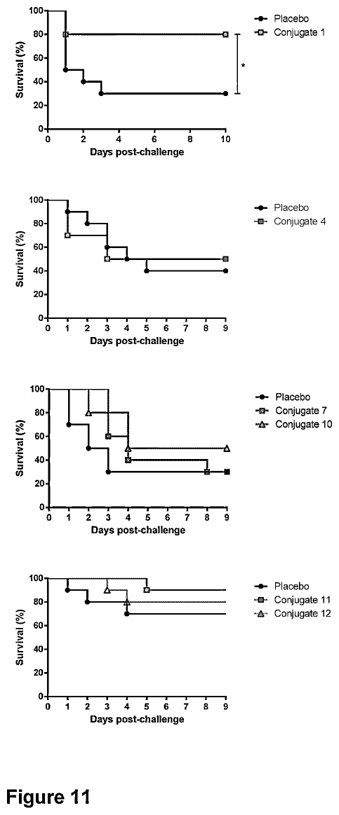

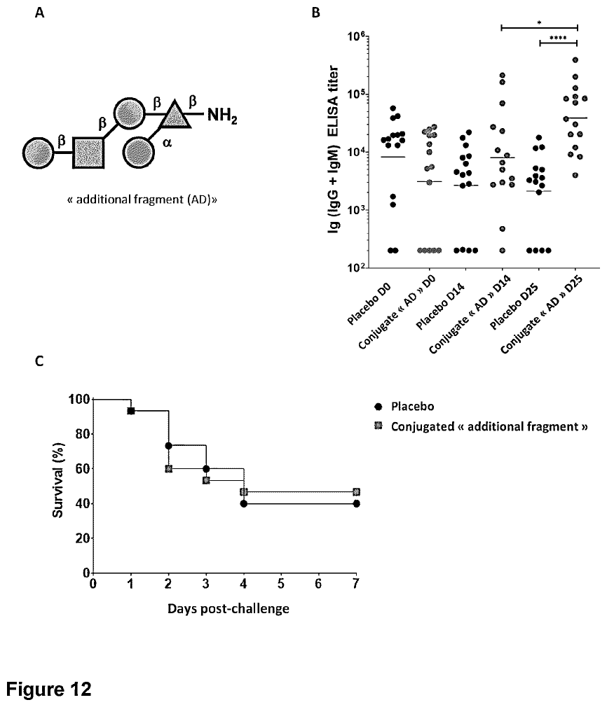

Experiments were conducted, wherein ELISA results show vaccine-induced antibody titers against the corresponding synthesized CPS epitope; see FIG. 9, FIG. 10, and FIG. 11 outline clinical data after S. suis challenge of immunized pigs. The levels of antibodies induced by the different fragments vary but the antibody isotype pattern is similar. Fragment 4 seems to be recognized by non-specific antibodies naturally present in the pigs (this is evidenced by a strong ELISA reaction in the placebo group against this fragment). This fragment is thus not suitable as a vaccine candidate. This finding demonstrates the importance of not only chemically design the right epitope but also that clinical evaluation in pigs is required to predict the real value of a fragment as a vaccine candidate. Indeed, not all conjugated CPS fragments are efficient in inducing protection (i.e., reduce clinical signs related to S. suis disease and/or reduce mortality levels). Indeed, the “additional fragment” induced significant levels of antibodies, but failed to protect pigs against clinical disease (FIG. 12). Therefore, claiming that a compound is a potential vaccine candidate should not be based on in vitro tests only. Based on animal (pig) studies, conjugates 1, 10, and 11 showed strong to partial protection and are thus promising targets for a vaccine formulation.

In embodiments of the invention, compounds of the following general formulae are provided: A0, B0, C0, D0, E0, F0, A, B, C, D, E, F, A01, B01, C01, D01, E01, F01, A1, B1, C1, D1, E1, F1, A02, B02, C02, D02, E02, and F02. Each of these general formulae is as described in detail herein. Also, in embodiments of the invention, compounds of the following chemical formulae are provided: A2, B2, C2, D2, E2, and F2. Each of these formulae is as described in detail herein.

In embodiments of the invention, the compounds are prepared by a process which comprises a chemical synthesis and/or a chemoenzymatic synthesis.

In embodiments of the invention, a glycoconjugate vaccine is provided which comprises a compound according to the invention and as described herein above. In other embodiments, the compound is conjugated with a carrier protein. In other embodiments, the carrier protein is CRM 197, BSA, or a protein from Streptococcus suis (S. suis). The carrier protein may also be any other suitable carrier protein as desired.

In embodiments of the invention, a vaccine formulation is provided, which comprises a compound according to the invention and as described herein above and an adjuvant. In other embodiments, the adjuvant is TiterMax Gold® or Montanide™ ISA 61 VG. The adjuvant may also be any other suitable adjuvant as desired.

In embodiments of the invention, a vaccine formulation is provided which comprises a glycoconjugate vaccine according to the invention and as described herein above and an adjuvant. In other embodiments, the adjuvant is TiterMax Gold® or Montanide™ ISA 61 VG. The adjuvant may also be any other suitable adjuvant as desired.

In embodiments of the invention, the vaccine formulation is commercially available.

In embodiments of the invention, the vaccine formulation is used in the production of livestock. In other embodiments, the vaccine formulation is used in the production of pigs. In other embodiments, the vaccine formulation is used in the swine production.

In embodiments of the invention, a process for preparing a glycoconjugate vaccine as described herein above is provided. In other embodiments, the process comprises a chemical synthesis and/or a chemoenzymatic synthesis.

In embodiments of the invention, there is provided a method of preventing a disease associated to Streptococcus suis (S. suis) in a mammal. The method comprises administering to the mammal a compound according to the invention as described herein above, a glycoconjugate vaccine according to the invention as described herein above, or a vaccine formulation according to the invention as described herein above. In other embodiments, the disease is associated to a serotype of S. suis selected from the group consisting of serotypes 1, 1/2, 2, 3, 9, and 14. In other embodiment, the disease is associated to serotype 2 of S. suis.

In embodiments of the invention, there is provided a use of a compound according to the invention and as described herein above, a glycoconjugate vaccine according to the invention and as described herein above, and a vaccine formulation according to the invention and as described herein above, each for preventing a disease associated to Streptococcus suis (S. suis) in a mammal. In other embodiments, the disease is associated to a serotype of S. suis selected from the group consisting of serotypes 1, 1/2, 2, 3, 9, and 14. In other embodiments, the disease is associated to serotype 2 of S. suis.

In embodiments of the invention, there is provided a use of a compound according to the invention and as described herein above, in the manufacture of a vaccine for preventing a disease associated to Streptococcus suis (S. suis) in a mammal. In other embodiments, the disease is associated to a serotype of S. suis selected from the group consisting of serotypes 1, 1/2, 2, 3, 9, and 14. In other embodiments, the disease is associated to serotype 2 of S. suis.

In embodiments of the invention, there is provided a compound according to the invention and as described herein above, a glycoconjugate vaccine according to the invention and as described herein above, and a vaccine formulation according to the invention and as described herein above, each for use in the prevention of a disease associated to Streptococcus suis (S. suis) in a mammal. In other embodiments, the disease is associated to a serotype of S. suis selected from the group consisting of serotypes 1, 1/2, 2, 3, 9, and 14. In other embodiments, the disease is associated to serotype 2 of S. suis.

In embodiments of the invention, the mammal is human or non-human.

As will be understood by a skilled person, other variations and combinations may be made to the various embodiments of the invention as described herein above.

Also, as will be understood by a skilled person, in embodiments of the invention, an OH group of any compound may be replaced by an SH group. In other embodiments of the invention, H in an OH group may be replaced by a lower alkyl group such a C1 to C3 alkyl group.

The scope of the claims should not be limited by the preferred embodiments set forth in the examples; but should be given the broadest interpretation consistent with the description as a whole.

The present description refers to a number of documents, the content of which is herein incorporated by reference in their entirety.

### REFERENCES

1. WO 2017/062558A1.
2. Zhang S, Sella M, Sianturi J, Priegue P, Shen D, Seeberger PH. Discovery of Oligosaccharide Antigens for Semi-Synthetic Glycoconjugate vaccine leads against Streptococcus suis serotypes 2, 3, 9 and 14. Angew Chem Int Ed Engl. 2021; 60(26):14679-14692. doi: 10.1002/anie.202103990. Epub 2021 May 19. PMID: 33852172; PMCID: PMC8252040.
3. Susaki, H.; Suzuki, K.; Ikeda, M.; Yamada, H.; Watanabe, H. K., Synthesis of Artificial Glycoconjugates of Arginine-Vasopressin and Their Antidiuretic Activities, Chem. Pharm. Bull. 1994, 42, 2090-2096.
4. Elsaidi, H. R. H.; Lowary, T. L., Effect of phenolic glycolipids from Mycobacterium kansasii on proinflammatory cytokine release. A structure-activity relationship study, Chem. Sci. 2015, 6, 3161-3172.
5. Deng, K.; Adams, M. M.; Gin, D. Y., Synthesis and Structure Verification of the Vaccine Adjuvant QS-7-Api. Synthetic Access to Homogeneous Quillaja saponaria Immunostimulants, J. Am. Chem. Soc. 2008, 130, 5860-5861.
6. Li, Z.; Gildersleeve, J. C., Mechanistic Studies and Methods To Prevent Aglycon Transfer of Thioglycosides, J. Am. Chem. Soc. 2006, 128, 11612-11619.
7. Bock, K.; Pedersen, C., A study of ¹³C,H coupling constants in hexopyranoses, J. Chem. Soc., Perkin Trans. 2 1974, 293-297.
8. Sun, J.; Han, X.; Yu, B., Synthesis of a typical N-acetylglucosamine-containing saponin, oleanolic acid 3-yl α-L-arabinopyranosyl-(1,2)-α-L-arabinopyranosyl-(1,6)-2-acetamido-2-deoxy-β-D-glucopyranoside, Carbohydr. Res. 2003, 338, 827-833.
9. Lu, X.; Kovac, P., Chemical Synthesis of the Galacturonic Acid Containing Pentasaccharide Antigen of the O-Specific Polysaccharide of Vibrio cholerae O139 and Its Five Fragments, J. Org. Chem. 2016, 81, 6374-6394.
10. Han, Z.; Chen, C.; Meng, C.; Gao, T.; Peng, P.; Chen, X.; Wang, F.; Cao, H., Chemoenzymatic synthesis of tumor-associated antigen N3 minor octasaccharide, J. Carbohydr. Chem. 2016, 35, 412-422.
11. Fang, J.-L.; Tsai, T.-W.; Liang, C.-Y.; Li, J.-Y.; Yu, C.-C., Enzymatic Synthesis of Human Milk Fucosides α1,2-Fucosyl para-Lacto-N-Hexaose and its Isomeric Derivatives, Adv. Synth. Catal. 2018, 360, 3213-3219.
12. Xu, Y.; Fan, Y.; Ye, J.; Wang, F.; Nie, Q.; Wang, L.; Wang, P. G.; Cao, H.; Cheng, J., Successfully Engineering a Bacterial Sialyltransferase for Regioselective α2,6-sialylation, ACS Catal. 2018, 8, 7222-7227.
13. Kondo, H.; Aoki, S.; Ichikawa, Y.; Halcomb, R. L.; Ritzen, H.; Wong, C.-H., Glycosyl Phosphites as Glycosylation Reagents: Scope and Mechanism, J. Org. Chem. 1994, 59, 864-877.
14. Zhang, Z.; Ollmann, I.; Ye, X.-S.; Wischnat, R.; Baasov, T.; Wong, C.-H., Programmable one-pot oligosaccharide synthesis, J. Am. Chem. Soc. 1999, 121, 734-753.
15. Xiong, C.; Feng, S.; Qiao, Y.; Guo, Z.; Gu, G., Synthesis and Immunological Studies of Oligosaccharides that Consist of the Repeating Unit of Streptococcus pneumoniae Serotype 3 Capsular Polysaccharide, Chem. Eur. J. 2018, 24, 8205-8216.
16. Lloyd, D.; Bylsma, M.; Bright, D. K.; Chen, X.; Bennett, C. S., Mild Method for 2-Naphthylmethyl Ether Protecting Group Removal Using a Combination of 2,3-Dichloro-5,6-dicyano-1,4-benzoquinone (DDQ) and β-Pinene, J. Org. Chem. 2017, 82, 3926-3934.
17. Liu, C.; Skogman, F.; Cai, Y.; Lowary, T., Synthesis of the ‘primer-adaptor’ trisaccharide moiety of Escherichia coli O8, O9, and O9a lipopolysaccharide, Carbohydr. Res. 2007, 342, 2818-2825.
18. Kelly, S. D.; Clarke, B. R.; Ovchinnikova, O. G.; Sweeney, R. P.; Williamson, M. L.; Lowary, T. L.; Whitfield, C., Klebsiella pneumoniae O1 and O2ac antigens provide prototypes for an unusual strategy for polysaccharide antigen diversification, J. Biol. Chem. 2019, 294, 10863-10876.
19. Huang, Y.-L.; Hung, J.-T.; Cheung, S. K.; Lee, H.-Y.; Chu, K.-C.; Li, S.-T.; Lin, Y.-C.; Ren, C.-T.; Cheng, T.-J.; Hsu, T.-L.; Yu, A. L.; Wu, C.-Y.; Wong, C.-H., Carbohydrate-based vaccines with a glycolipid adjuvant for breast cancer. Proc. Natl. Acad. Sci. U.S.A. 2013, 110, 2517-2522., Proc. Natl. Acad. Sci. U.S.A 2013, 110, 2517-2522.
20. Kamath, V. P.; Diedrich, P.; Hindsgaul, O., Use of diethyl squarate for the coupling of oligosaccharide amines to carrier proteins and characterization of the resulting neoglycoproteins by MALDI-TOF mass spectrometry, Glycoconj. J. 1996, 13, 315-319.

1. A compound of general formula selected from the group consisting of: A0, B0, C0, D0, E0, and F0 below 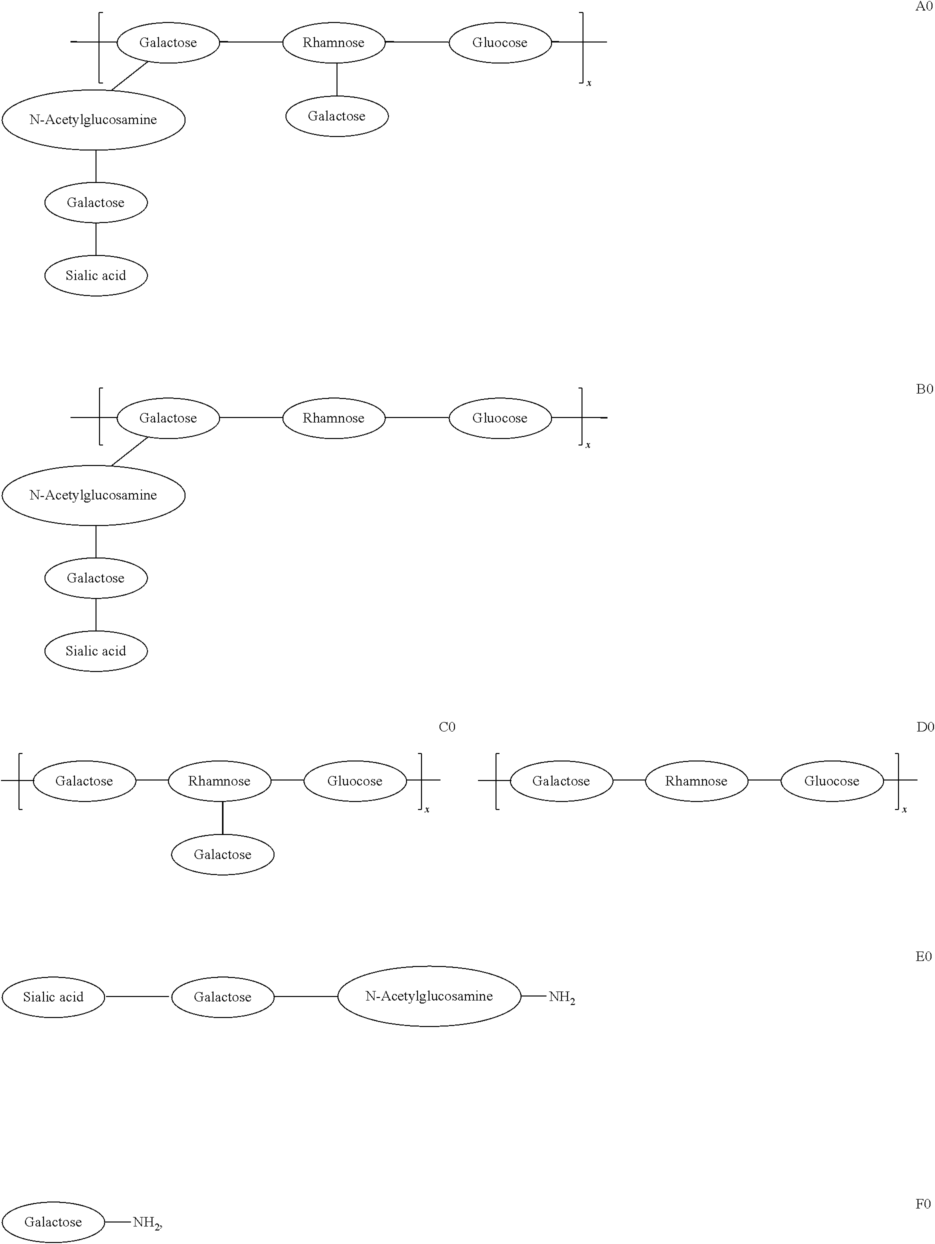
wherein:
x in A0-D0 is an integer selected from 1 to 6; preferably x is an integer selected from 1 to 3; and
a link between consecutive sugar moieties in the compound is a or p.

2. A compound of general formula selected from the group consisting of: A, B, C, D, E, and F below 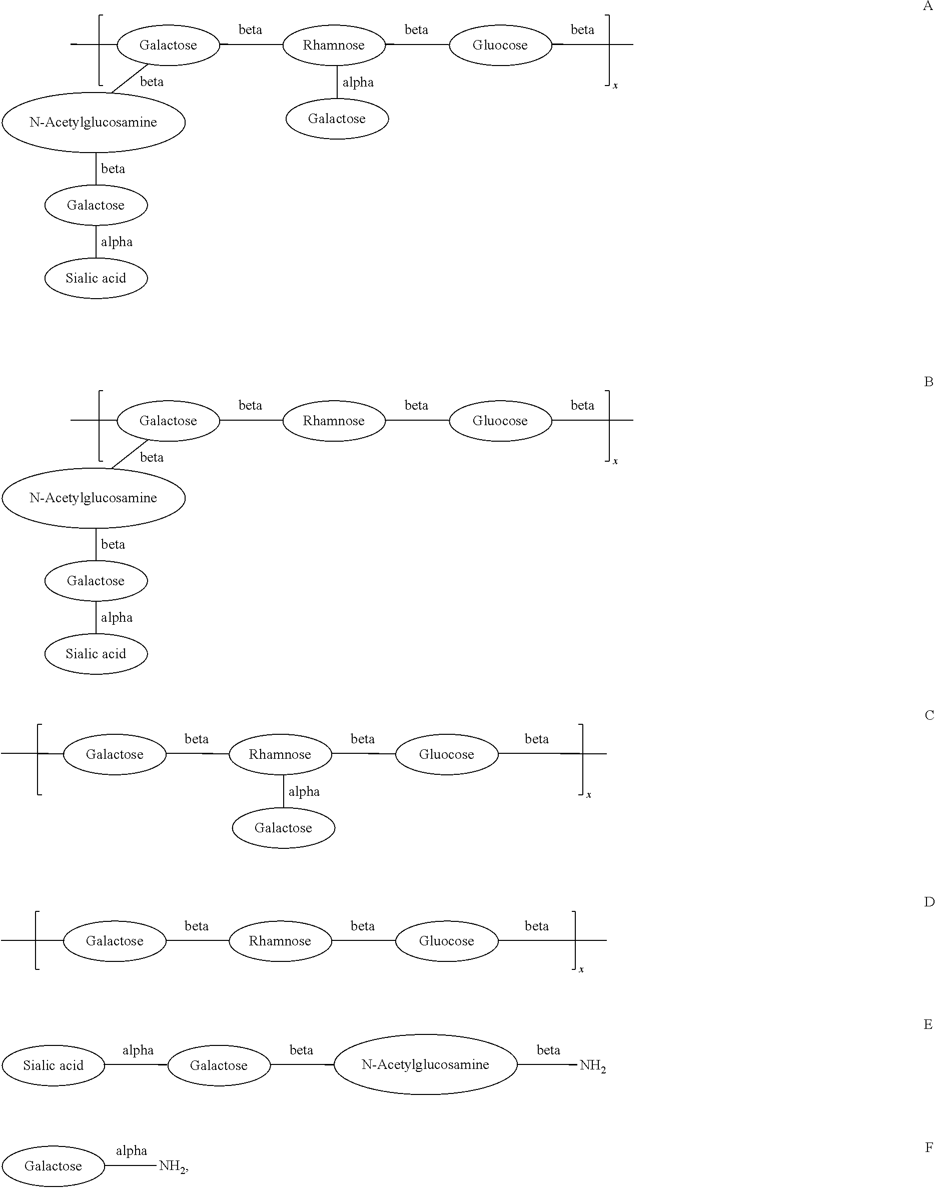
wherein x in A-D is an integer selected from 1 to 6; preferably x is an integer selected from 1 to 3.

3. A compound of general formula selected from the group consisting of: A01, B01, C01, D01, E01, and F01 below 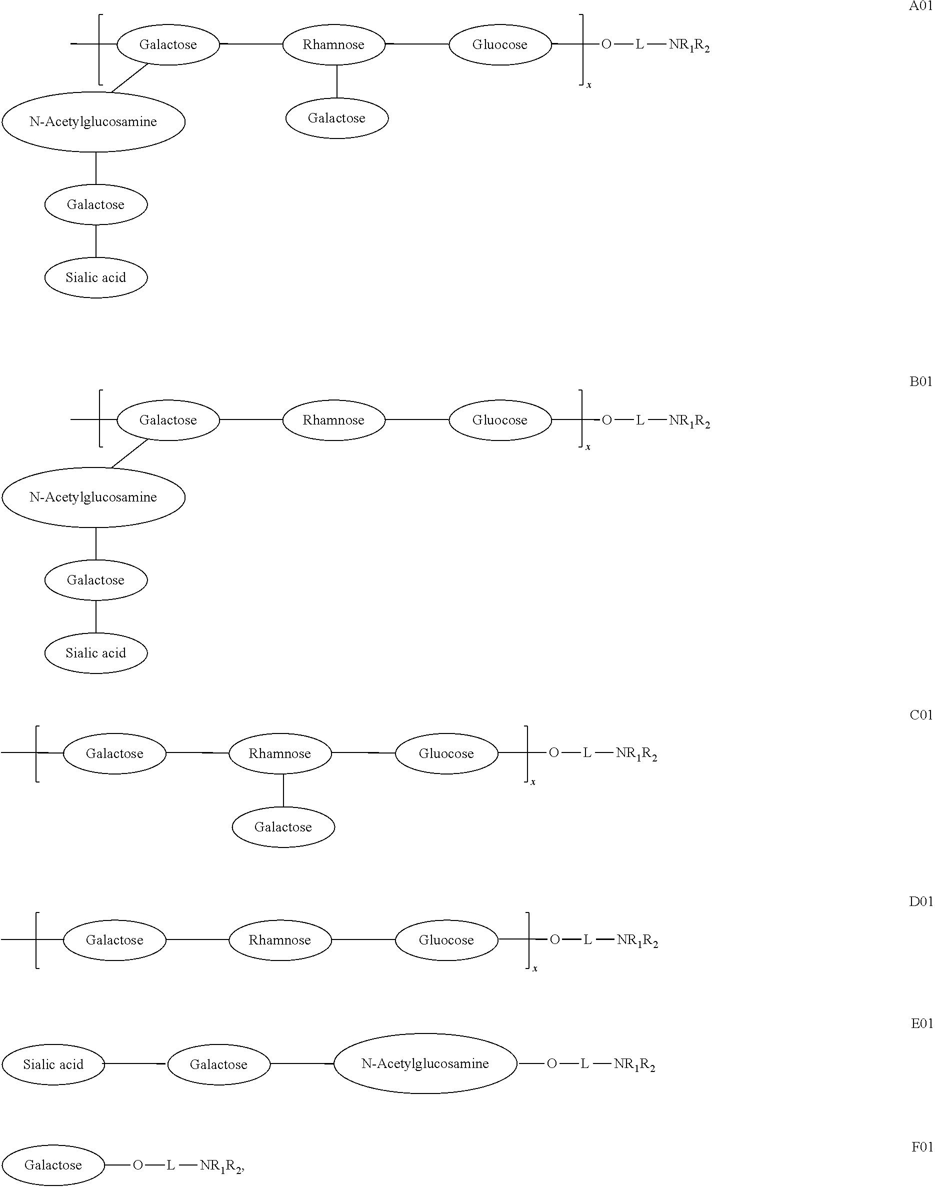
wherein:
x in A01-D01 is an integer selected from 1 to 6; preferably x is an integer selected from 1 to 3;
a link between consecutive sugar moieties in the compound is a or p;
L is a C1 to C12 linear, branched, or cyclic alkyl; preferably L is a linear alkyl; and
R₁ and R₂ are each independently H or a C1 to C6 linear, branched, or cyclic alkyl; or R₁ and R₂ together with N form a 3 to 6-member ring.

4. A compound of general formula selected from the group consisting of: A1, B1, C1, D1, E1, and F1 below 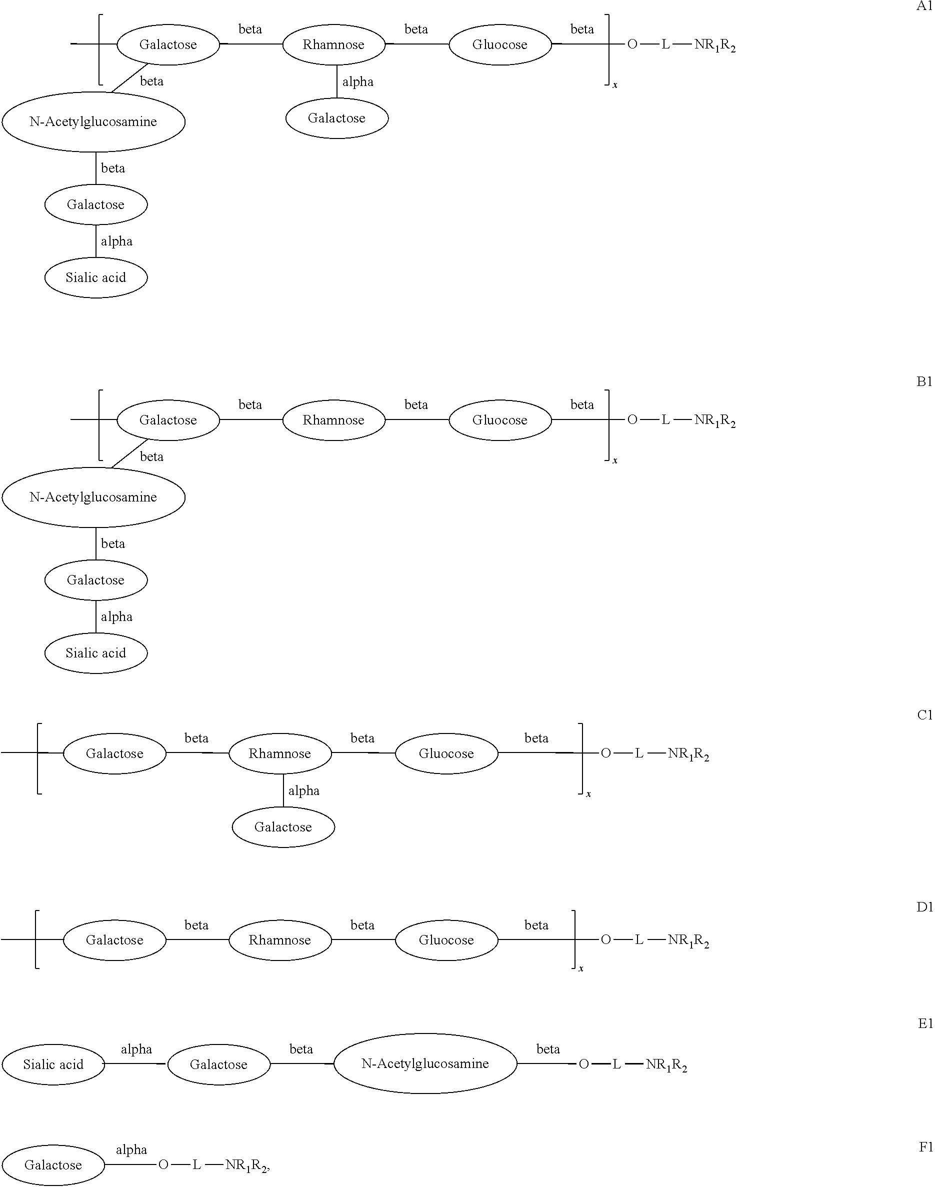
wherein:
x in A1-D1 is an integer selected from 1 to 6; preferably x is an integer selected from 1 to 3;
L is a C1 to C12 linear, branched, or cyclic alkyl; preferably L is a linear alkyl; and
R₁ and R₂ are each independently H or a C1 to C6 linear, branched, or cyclic alkyl; or R₁ and R₂ together with N form a 3 to 6-member ring.

5. A compound of general formula selected from the group consisting of: A02, B02, C02, D02, E02, and F02 below 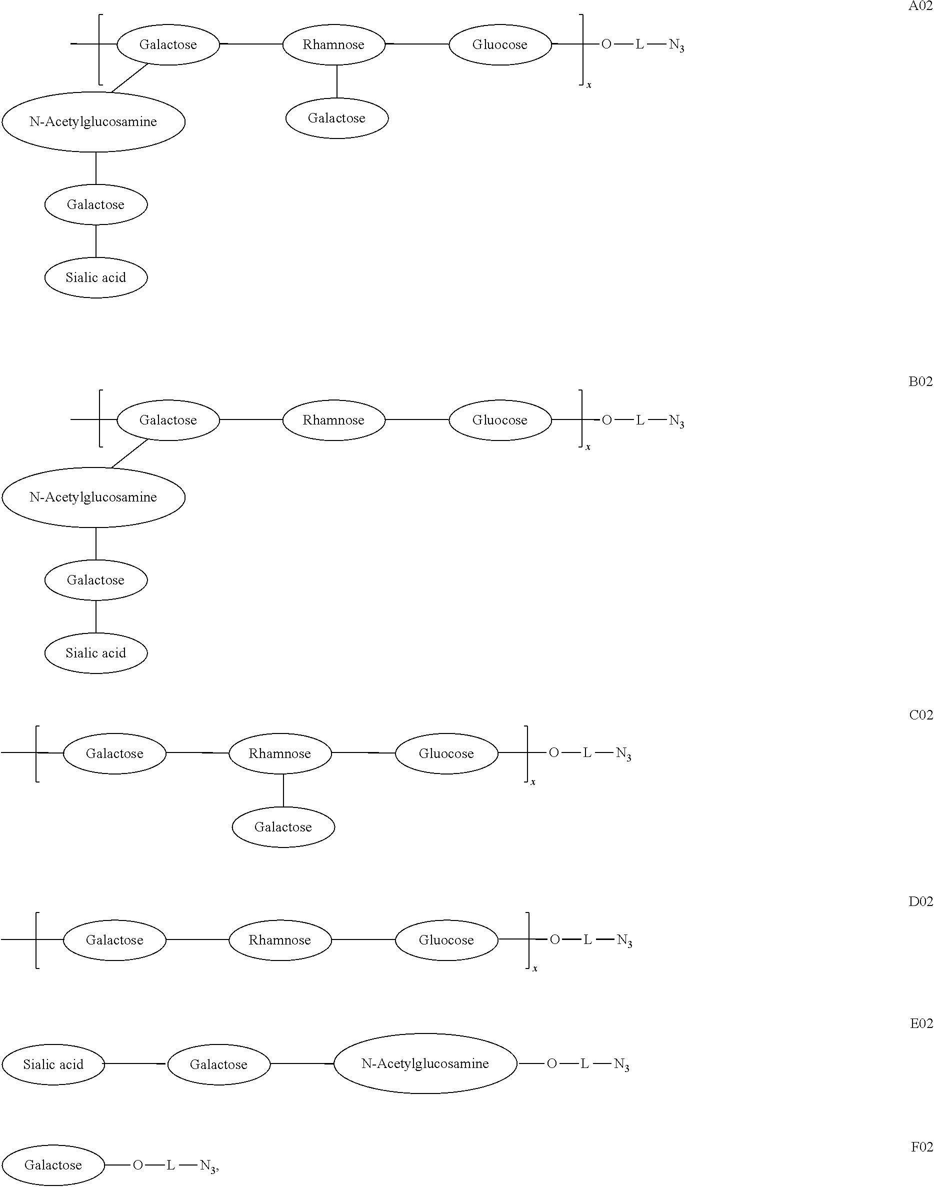
wherein:
x in A02-D02 is an integer selected from 1 to 6; preferably x is an integer selected from 1 to 3;
a link between consecutive sugar moieties in the compound is a or p;
L is a C1 to C12 linear, branched, or cyclic alkyl; preferably L is a linear alkyl; and
R₁ and R₂ are each independently H or a C1 to C6 linear, branched, or cyclic alkyl; or R₁ and R₂ together with N form a 3 to 6-member ring.

6. A compound of general formula selected from the group consisting of: A2, B2, C2, D2, E2, and F2 below 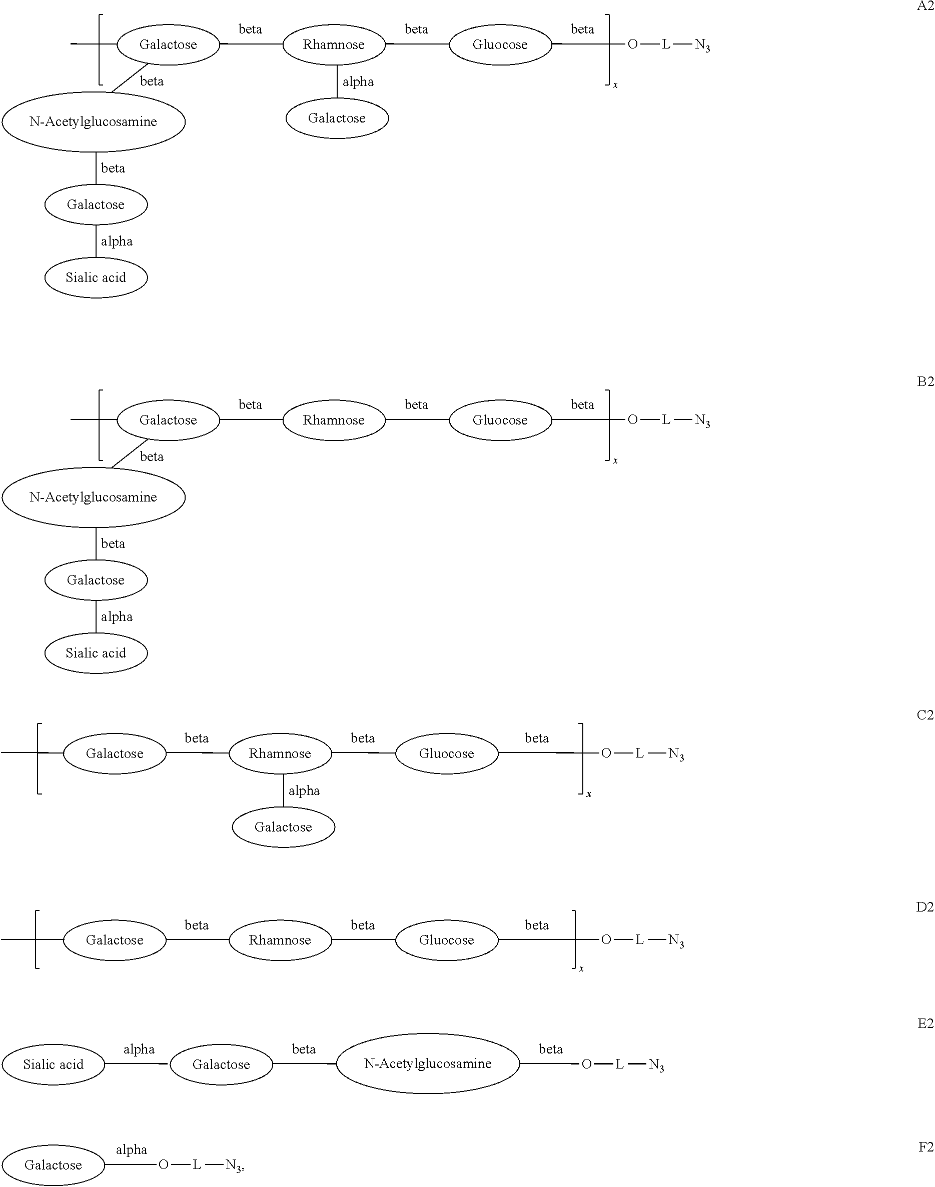
wherein:
x in A2-D2 is an integer selected from 1 to 6; preferably x is an integer selected from 1 to 3; and
L is a C1 to C12 linear, branched, or cyclic alkyl; preferably L is a linear alkyl.

7. A compound selected from the group consisting of: 1, 4, 7, and 10-14 below

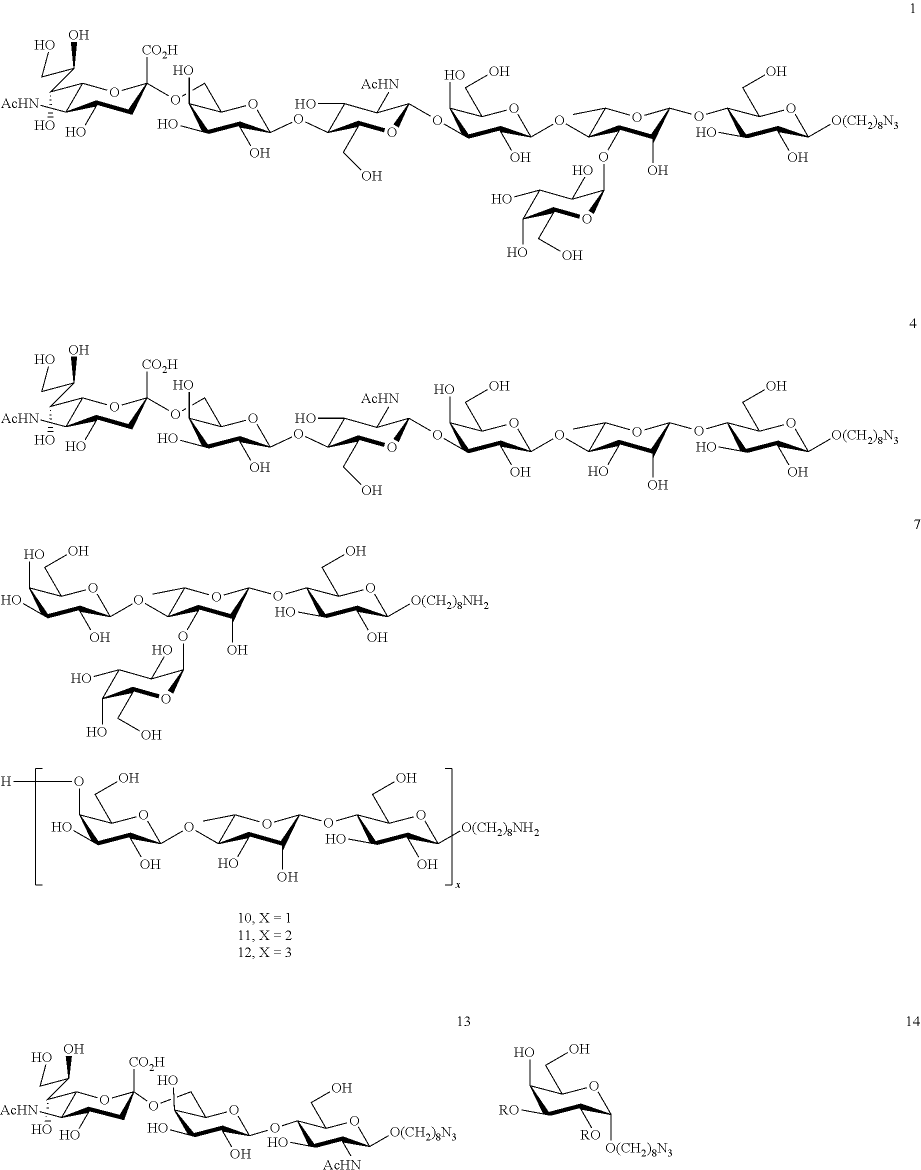

8. A compound selected from the group consisting of: 15, 16, 17, and 18-22 below

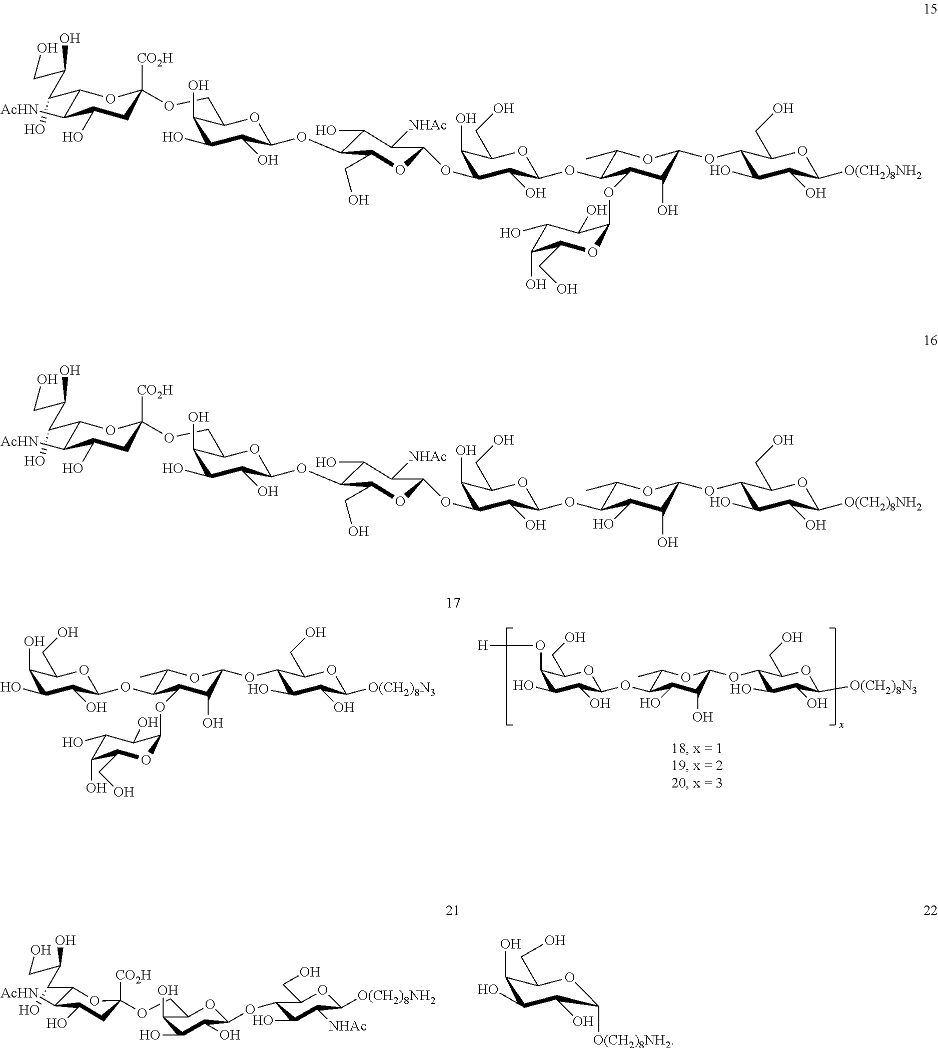

9. A compound according to any one of claims 1 to 8, which is prepared by a process comprising: a chemical synthesis, a chemoenzymatic synthesis, or a combination of both chemical synthesis and chemoenzymatic synthesis.

10. A glycoconjugate vaccine comprising a compound as defined in any one of claims 1 to 8, wherein the compound is conjugated with a carrier protein; preferably the carrier protein is CRM-197, BSA, a protein from Streptococcus suis (S. suis), or another suitable carrier protein.

11. A vaccine formulation comprising a compound as defined in any one of claims 1 to 8 and an adjuvant; preferably the adjuvant is TiterMax Gold®, Montanide™ ISA 61 VG, or another suitable adjuvant.

12. A vaccine formulation comprising a glycoconjugate vaccine as defined in claim 10 and an adjuvant; preferably the adjuvant is TiterMax Gold®, Montanide™ ISA 61 VG, or another suitable adjuvant.

13. A vaccine formulation according to claim 11 or 12, which is commercially available.

14. A vaccine formulation according to any one of claims 11 to 13, which is used in the production of livestock.

15. A process for preparing a glycoconjugate vaccine as defined in claim 10 or a vaccine formulation as defined in any one of claims 11 to 13, comprising: a chemical synthesis, a chemoenzymatic synthesis, or a combination of both chemical synthesis and chemoenzymatic synthesis.

16. A method of preventing a disease associated to Streptococcus suis (S. suis) in a mammal, the method comprising administering to the mammal a compound as defined in any one of claims 1 to 8, a glycoconjugate vaccine as defined in claim 10, or a vaccine formulation as defined in any one of claims 11 to 13; preferably the disease is associated to a serotype of S. suis selected from the group consisting of serotypes 1, 1/2, 2, 3, 9, and 14; more preferably the disease is associated to serotype 2 of S. suis.

17. Use of a compound as defined in any one of claims 1 to 8, a glycoconjugate vaccine as defined in claim 10, or a vaccine formulation as defined in any one of claims 11 to 13, for preventing a disease associated to Streptococcus suis (S. suis) in a mammal; preferably the disease is associated to a serotype of S. suis selected from the group consisting of serotypes 1, 1/2, 2, 3, 9, and 14; more preferably the disease is associated to serotype 2 of S. suis.

18. Use of a compound as defined in any one of claims 1 to 8, in the manufacture of a vaccine for preventing a disease associated to Streptococcus suis (S. suis) in a mammal; preferably the disease is associated to a serotype of S. suis selected from the group consisting of serotypes 1, 1/2, 2, 3, 9, and 14; more preferably the disease is associated to serotype 2 of S. suis.

19. A compound as defined in any one of claims 1 to 8, a glycoconjugate vaccine as defined in claim 10, or a vaccine formulation as defined in any one of claims 11 to 13, for use in the prevention of a disease associated to Streptococcus suis (S. suis) in a mammal; preferably the disease is associated to a serotype of S. suis selected from the group consisting of serotypes 1, 1/2, 2, 3, 9, and 14; more preferably the disease is associated to serotype 2 of S. suis.

20. A method or use or compound for use according to any one of claims 16 to 19, wherein the mammal is human or non-human.
